(19) United States
(12) Patent Application Publication
(10) Pub. No.: US 2025/0228588 A1
(43) Pub. Date: Jul. 17, 2025

(54) DEVICES, SYSTEMS, AND METHODS FOR PERFORMING THROMBECTOMY PROCEDURES

(71) Applicant: THE BOARD OF TRUSTEES OF THE LELAND STANFORD JUNIOR UNIVERSTIY, Stanford, CA (US)

(72) Inventors: Renee Zhao, Stanford, CA (US); Yilong Chang, Stanford, CA (US); Shuai Wu, Stanford, CA (US); Jeremy J. Heit, Los Altos, CA (US); Paul G. Yock, Atherton, CA (US)

(21) Appl. No.: 19/008,511

(22) Filed: Jan. 2, 2025

Related U.S. Application Data
(63) Continuation-in-part of application No. 18/939,316, filed on Nov. 6, 2024.
Application No. 18/939,316 is a continuation of application No. PCT/US23/21388, filed on May 8, 2023.
(60) Provisional application No. 63/339,504, filed on May 8, 2022.
(60) Provisional application No. 63/418,449, filed on Oct. 21, 2022.
(60) Provisional application No. 63/453,152, filed on Mar. 19, 2023.
(60) Provisional application No. 63/618,237, filed on Jan. 5, 2024.

(51) Int. Cl. A61B 17/3207; A61B 17/22

(52) U.S. Cl. CPC A61B 17/320758; A61B 2017/22079

(57) ABSTRACT

Thrombectomy devices are provided that include an aspiration catheter and a spinner device movable axially within the aspiration catheter. The spinner device includes a rotation shaft including a proximal end coupled to a motor configured to spin the shaft, a distal end carrying a spinner head movable axially within the aspiration catheter and configured to generate localized suction when the shaft rotates. The spinner device may also include an outer sleeve surrounding the rotation shaft at least partially along its length to protect the aspiration catheter. The spinner device and aspiration catheter include cooperating stops that limit advancement of the spinner device to prevent the spinner head from being exposed beyond a short distance beyond a distal end of the aspiration catheter, e.g., a wing-stopper provided on one of the rotation shaft, spinner head, and outer sleeve, and a corresponding ring within the distal end of the aspiration catheter.

### PRIORITY

This patent application claims benefit as a continuation-in-part of U.S. patent application Ser. No. 18/939,316, titled “DEVICES, SYSTEMS, AND METHODS FOR PERFORMING THROMBECTOMY PROCEDURES,” filed Nov. 6, 2024, which is a continuation of International application no. PCT/US2023/021388, filed May 8, 2023, which claims benefit of U.S. provisional applications Ser. Nos. 63/339,504, filed May 8, 2022, 63/418,449, filed Oct. 21, 2022, and 63/453,152, filed Mar. 19, 2023. This patent application also claims the benefit of U.S. provisional patent application No. 63/618,237, titled “DEVICES, SYSTEMS, AND METHODS FOR PERFORMING THROMBECTOMY PROCEDURES,” filed Jan. 5, 2024. Each of these patent applications are herein incorporated by reference in their entirety.

### STATEMENT REGARDING FEDERALLY SPONSORED RESEARCH AND DEVELOPMENT

This invention was made with Government support under Contract 2145601 awarded by the National Science Foundation. The Government has certain rights in the invention.

### TECHNICAL FIELD

The present application relates to medical devices and, more particularly, to thrombectomy devices, and to systems and methods for performing thrombectomy or other medical procedures using such devices.

### BACKGROUND

Thromboembolism, usually referred as blood clot, is the result of blood coagulation in veins or arteries that disrupts the normal flow of blood to a part of the body. Blood clots can occur at many locations in the body; for example, a clot located in a deep vein in a leg or arm may cause deep vein thrombosis (DVT), and a clot may travel from a deep vein to a lung to cause a pulmonary embolism (PE), or from an artery to the brain to cause a stroke, which are life-threatening conditions. Depending on the location within a subject's vasculature, thrombi can result in venous thromboembolism, pulmonary embolism, cerebrovascular stroke, peripheral artery occlusion, coronary thrombus, and/or acute myocardial infarction.

Based on the CDC, venous thromboembolism affects around 900,000 Americans each year. As many as 100,000 people die of blood clots each year. One of four people who have a PE dies without warning. More sadly, PE is a leading cause of death in a woman during pregnancy or just after having a baby. Blood clots are also a leading cause of death in people with cancer after the cancer itself. It costs up to ten billion dollars each year in the US to address problems related to blood clots, and the treatment can be as much as $15,000 to $20,000 per person and often results in readmission to the hospital.

Acute Ischemic Stroke (AIS) is the leading cause of disability and the fifth leading cause of death in the United States. AIS results from blockage or interruption of blood flow within a cervical or cerebral artery, and this lack of blood flow to the brain may result in irreversible brain injury (core infarction) or impaired neuronal function in ischemic, but potentially salvageable, brain tissue (penumbra). AIS may be treated with intravenous thrombolysis within 3-4.5 hours of symptom onset, but fewer than five percent of AIS patients reach medical care within this time frame.

Percutaneous thrombectomy is a minimally invasive interventional treatment, during which the surgeon inserts a catheter into the patient's blood vessel to remove the blood clot and restore blood flow to the affected area. There are two commonly used mechanical thrombectomy technologies to remove large clots: (i) aspiration thrombectomy, where a continuous vacuum aspiration is induced through a guide catheter to suction out the clot; (ii) stent retriever thrombectomy, where a mesh tube is used pull out the clot, e.g., as shown in FIG. 2A. However, the use of stent retrievers and aspiration devices is always associated with a risk of thrombus fragmentation during which the big clot may break into small pieces (100 μm˜1000 μm) and travel downstream in the blood vessel, potentially blocking the blood flow at multiple other locations and/or leading to new life-threatening emboli that may require emergent open surgeries.

More recently, endovascular thrombectomy using aspiration or a stent-retriever device has been shown to be an effective treatment for AIS that involves large vessel occlusion (AIS-LVO) of the internal carotid, proximal middle cerebral, proximal anterior cerebral arteries, or basilar artery up to twenty-four hours from symptom onset. Thrombectomy has led to a marked improvement in AIS-LVO patient outcomes, and it has become the standard of care for AIS patients with an AIS-LVO.

Despite the recent advancements in AIS treatment, there are significant gaps in knowledge and available devices that limit optimal treatment of AIS treatments. Current thrombectomy techniques fail to restore any or insufficient blood flow in about fifteen percent (15%) of patients after multiple passes, with aspiration methods having a failure rate of about twenty-five to thirty three percent (25-33%). Common reasons for failed thrombectomy include clots with high fibrin content, clot fragmentation that prevents complete removal, and clots that are resistant to modern thrombectomy devices. In addition, recent data indicate that in order to be maximally effective, thrombectomy should restore about ninety-five to one hundred percent (95-100%) of the blood flow distal to the site of arterial blockage, and this blood flow should be restored within one thrombectomy attempt (“first pass effect”) to maximize the likelihood of achieving a good outcome. However, the first pass effect is achieved in fewer than about fifty percent (50%) of patients who undergo thrombectomy, and there is a substantial need to develop new thrombectomy devices to improve the thrombectomy process.

Therefore, improved devices and methods for performing thrombectomy procedures would be useful.

### SUMMARY

The present application is directed to medical devices and, more particularly to thrombectomy devices, and to systems and methods for performing thrombectomy or other medical procedures using such devices. In one example, the devices may include a spinner device introduced through a catheter or other tubular member (or directly into a body lumen) that mechanically reduces or compresses the volume and/or separates components of the clot (e.g., red blood cells, from fibrin or other residual fiber material) and/or dissolves or partially dissolves the clot, e.g., by coupled suction-induced compression and shear load applied to the clot by the spinner. That is, it should be understood that the terms “reducing”, “reduce”, or “reduces” with respect to a clot, include any manner in which a clot volume is reduced, including by separating components of the clot, e.g., red blood cells, from fibrin or other residual fiber material and/or compressing the clot and/or dissolving or partially dissolving the clot and/or reducing the volume of the residual clot material, e.g., to facilitate removal of the residual material. For example, the spinner may rotate rapidly to squeeze out red blood cells (RBCs) in a clot leaving a compacted fibrin fiber network, which may then be captured by the spinner tip, aspirated into the catheter, and/or otherwise removed from the vessel.

In addition or alternatively, suction may be applied to enhance reducing the clot and/or preventing fragmentation, suction generated by the spinner or another source applying a vacuum to the treatment site. Optionally, additional devices may be provided that may engage the clot to enhance spinning, e.g., one or more wires extending from the spinner or another device introduced into the treatment site separately. Optionally, a jet of saline or other fluid may also be directed into the treatment site to spin and/or otherwise enhancing dissolving the clot. Thus, the devices and systems herein may help to dissolve clots, reduce the size of blood clots, reduce thrombus fragmentation, and/or prevent large segmental debris from traveling downstream, which may reduce risks during interventional/endovascular procedures.

In accordance with one example, a thrombectomy device is provided that includes an elongated shaft comprising a proximal end configured to be coupled to a controller to spin the shaft, a distal end sized for introduction into a body lumen of a patient, and a longitudinal axis extending therebetween, the shaft configured to rotate about the axis; and a spinner member or element on the distal end configured to generate localized suction and/or shear force adjacent the distal end when the shaft rotates to reduce or dissolve a clot, reduce clot size, and/or prevent fragmentation of a clot within the body lumen. For example, the spinner tip may include an annular body extending distally from the distal end such that an opening communicating with a cavity within the annular body may be positioned adjacent the clot to apply the localized suction and/or shear force to the clot. Optionally, the spinner tip may include one or more blades or other external features on the annular body and/or one or more slits or other openings in the wall of the annular body, e.g., to enhance the localized suction that is generated at the opening and within the cavity.

In accordance with another example, a thrombectomy device is provided that includes a catheter or other tubular member including a proximal end, a distal end sized for introduction into a body lumen adjacent clot, and a lumen extending from the proximal end to an outlet in the distal end; an elongated shaft comprising a proximal end configured to be coupled to a controller to spin the shaft, a distal end sized for introduction into the lumen, and a longitudinal axis extending therebetween, the shaft configured to rotate about the axis; optionally, a sleeve surrounding the shaft; and a spinner tip on the shaft distal end configured to generate localized suction and/or shear force adjacent the outlet when the shaft rotates to reduce clot size of clot within a body lumen adjacent the outlet, e.g., by applying localized compression and/or shear forces to the clot to squeeze out red blood cells (RBCs) leaving a compacted fibrin fiber network.

Optionally, the fibrin network or other residual material may then be removed, e.g., aspirated into the tubular member and/or captured by the spinner tip, which may be withdrawn to remove the residual material.

In accordance with still another example, a system is provided for performing a thrombectomy procedure that includes a tubular member including a proximal end, a distal end sized for introduction into a body lumen adjacent clot, and a lumen extending from the proximal end to an outlet in the distal end; an elongated shaft comprising a proximal end, a distal end sized for introduction into the lumen, and a longitudinal axis extending therebetween; a motor and/or controller coupled to the proximal end of the shaft to rotate the shaft about the axis; and a spinner tip on the shaft distal end configured to generate localized suction and/or shear force adjacent the outlet when the shaft rotates to reduce the size of clot within a body lumen adjacent the outlet.

In accordance with yet another example, a method is provided for performing thrombectomy that includes introducing a spinner device into a body lumen adjacent a target blood clot; and rotating the spinner device to generate localized suction to dissolve the clot, reduce clot size, and/or prevent fragmentation of the clot.

In accordance with another example, a tethered biomedical device is provided that includes an elongated tether having a proximal end and a distal end, and a tool head disposed on the distal end of the tether. The tether is an elongated, flexible member, such as a shaft, cable, tubular member, guidewire, or similar apparatus. The tether is configured to be advanced along a pathway within the body, including body lumens and/or body cavities and, optionally, one or more catheters, introducer sheaths, guidewires, or other delivery devices introduced into any such pathway. For example, the tether may be navigated by a combination of pushing, pulling, and rotating to steer the device to a target location, e.g., within a catheter or other delivery device. The tether is also configured to be rotated by a rotational driver to spin the tool head.

The tool head may be configured to perform ablation of body tissue and/or create suction, e.g., to reduce or dissolve clots. For example, the tool head may include a tubular body having a lumen extending axially along a central axis of the tubular body. Optionally, the tool head may include one or more blades disposed on, and extending radially outward from, the exterior surface of the tool head, which may enhance the suction and/or ablative function of the tool head.

In one example, the blades may be straight, raised ribs on the exterior surface of the tubular body and aligned parallel to the central axis of the tubular body.

In another example, the tubular body may include a plurality of holes through the wall of the tubular body. The holes in the tubular body may improve the localized suction capability of the tethered biomedical device. The holes may be slits, apertures, or other through holes in the tubular body, which may help facilitate clot removal. In the case that the device has blades, the holes may be between the blades. The holes are angularly spaced around tubular body. For instance, the tubular body may have 2 holes angularly spaced by 180° around the tubular body, or 4 holes angularly spaced by 90° around the tubular body.

In examples in which the tool head has both blades and holes, the holes may be positioned between the blades. For example, the tubular body may have two blades and two holes positioned between the adjacent blades, or the tubular body may have four blades and four holes spaced between the adjacent blades.

In accordance with still another example, a method is provided for using a tethered biomedical device. The tethered medical device is introduced into a patient's body via a small incision. Optionally, an introducer is inserted into the incision and the tool head is inserted into the body through the introducer. The tethered medical device is navigated to position the tool head proximate a target location within the body by pushing the tether and steering the tether through a pathway within the body, including body lumens and/or body cavities. Once the tool head is advanced to the target location, the tool head is used to perform a biomedical procedure, such as a diagnostic or treatment procedure. After performing the biomedical procedure, the tethered medical device is retracted from the body by retracting the tether, same or similar to advancing the device to the target location, except in the opposite direction.

In another aspect of the method, the biomedical procedure is an ablation procedure performed by spinning the tool head while bearing the tool against body tissue at the target location to remove the body tissue. The tool head may be translated, and its orientation adjusted, to position the tool head to ablate the body tissue by manipulating the tether. The tool head is spun by spinning the tether using the rotational driver. In still another aspect, the body tissue is an occlusion, such as thrombus and/or plaque, within a blood vessel.

In another aspect, the method may include using the tethered medical device to capture and remove an object (e.g., material such as a blood clot or tissue ablated by the tool head) at the target location. In this aspect, the tool head is positioned proximate the object and then the tool head is rotated by spinning the tether using the rotational driver. The spinning tool head creates a suction (a low-pressure zone) within the lumen of the tubular body which pressurizes and/or compresses the object toward a distal face of the tool head. The object may be sucked into the lumen of the tubular body, or pressed close to the tool head. The tool head may then be retracted along the pathway by pulling the tether, e.g., while spinning the tool head or with the tool head stationary, to pull the object along the pathway and out of the body.

Other aspects and features of the present invention will become apparent from consideration of the following description taken in conjunction with the accompanying drawings.

As mentioned, the devices may include a spinner device introduced through an aspiration catheter or other tubular member (or directly into a body lumen) that mechanically reduces or compresses the volume and/or separates components of the clot (e.g., red blood cells, from fibrin or other residual fiber material) and/or dissolves or partially dissolves the clot, e.g., by coupled suction-induced compression and shear load applied to the clot by the spinner. The devices and systems herein may include positioning mechanisms, e.g., one-way or two-way stopping or locking mechanisms, that may prevent exposure of a spinner head from an aspiration or other delivery catheter into a blood vessel or body cavity. These locking mechanisms may enhance operation and/or safety during use of the spinner head to dissolve or reduce clot, unlike conventional devices, which may require an active component to be deployed directly into a blood vessel and/or into or through the clot during a thrombectomy procedure. For example, the locking mechanisms herein may provide greater control in positioning the spinner head, e.g., within or otherwise adjacent an outlet of a delivery catheter, which may maximize localized suction and/or prevent exposure of the spinner head when it is activated.

In accordance with one example, a thrombectomy device is provided that includes a rotation shaft comprising a proximal end configured to be coupled to a controller to spin the shaft, a distal end carrying a spinner head configured to generate localized suction and/or shear force when the shaft rotates to reduce or dissolve a clot, reduce clot size, and/or prevent fragmentation of a clot within the body lumen. For example, the spinner head may include an annular body extending distally from the distal end such that an opening communicating with a cavity within the annular body may be positioned adjacent the clot to apply the localized suction and/or shear force to the clot. Optionally, the spinner head may include one or more blades or other external features on the annular body and/or one or more slits or other openings in the wall of the annular body, e.g., to enhance the localized suction that is generated at the opening and within the cavity.

The spinner device includes one or more stops that limit advancement of the spinner device within an aspiration catheter or other tubular member, e.g., to prevent the spinner head from being exposed beyond a distal end of the aspiration catheter and/or to ensure the spinner head is positioned to achieve maximum dissolving, debulking, and/or removal of clot. In various examples, the one or more stops may include a wing-stopper provided on one of the rotation shaft, spinner head, and outer sleeve, configured to interact with a corresponding ring or other within the distal end of the aspiration catheter. Alternatively, other stops may be provided on or adjacent the spinner head and the catheter to limit distal movement of the spinner head to a distal-most position. In a further alternative, one or more stops may be provided on the hub of the catheter and/or shaft of the spinner device to limit distal movement of the spinner head.

In addition or alternatively, the spinner device may include a two-way locking mechanism that axially fixes the spinner device relative to the tubular member, e.g., once the spinner reaches its distal-most position when the one or more stops interact. For example, two sets of distal stops may be provided on or adjacent the spinner head and/or catheter distal end that limit distal advancement of the spinner head and then axially fix the spinner head to prevent subsequent proximal movement, e.g., until the spinner head is released. Alternatively, a distal stop may be provided on or adjacent the spinner head and/or catheter distal end to limit distal advancement of the spinner head and a proximal stop may be provided, e.g., on the proximal hub of the catheter and/or the shaft of the spinner device, to axially fix the spinner head to prevent subsequent proximal movement, e.g., until the proximal stop is released.

In accordance with still another example, a system is provided for performing a thrombectomy procedure that includes an aspiration catheter and a spinner device movable axially within the aspiration catheter. The spinner device includes a rotation shaft including a proximal end coupled to a motor configured to spin the shaft, a distal end carrying a spinner head movable axially within the aspiration catheter and configured to generate localized suction when the shaft rotates. The spinner device may also include an outer sleeve surrounding the rotation shaft at least partially along its length to protect. The spinner device and aspiration catheter include cooperating stops that limit advancement of the spinner device to prevent the spinner head from being exposed beyond a distal end of the aspiration catheter, e.g., a wing-stopper provided on one of the rotation shaft, spinner head, and outer sleeve, and a corresponding ring within the distal end of the aspiration catheter.

In accordance with yet another example, a method is provided for performing thrombectomy that includes introducing a distal end of an aspiration catheter or other tubular member into a body lumen and then advanced to a point that is adjacent a target blood clot; advancing a spinner head of a spinner device within the tubular member to position the spinner head adjacent the distal end of the tubular member, one or more cooperating stops limiting distal movement of the spinner head, e.g., to prevent the spinner head from being exposed beyond a distal end of the tubular member. The spinner head is rotated to generate localized suction adjacent the distal end of the tubular member to dissolve the clot, reduce clot size, and/or prevent fragmentation of the clot.

Other aspects and features of the present invention will become apparent from consideration of the following description taken in conjunction with the accompanying drawings.

### BRIEF DESCRIPTION OF THE DRAWINGS

It is believed the present invention will be better understood from the following description of certain examples taken in conjunction with the accompanying drawings, in which like reference numerals identify the same elements and in which:

FIG. 1A shows an example of a blood clot in a blood vessel.

FIG. 1B is a schematic showing how blood clots at different locations in a body can cause deep vein thrombosis (leg/arm), pulmonary embolism (lung), and stoke (brain).

FIGS. 2A and 2B show an example of mechanical thrombectomy using a stent retriever, showing the thrombus fragmentation that may result during such a procedure.

FIG. 3 shows an example of a thrombectomy device including a catheter or sheath and a spinner connected to a flexible shaft insertable into the catheter.

FIG. 4 shows an exemplary spinner tip attached to a distal end of a shaft, which may be included in the device of FIG. 3.

FIGS. 4A-4C show exemplary spinner tips that may be included in the device of FIG. 3.

FIG. 4D shows the spinner tip of FIG. 4C being loaded with a drug delivery member.

FIGS. 5A and 5B are cross-sectional views of a blood vessel showing an exemplary method for dissolving a blood clot using the device of FIG. 3.

FIGS. 6A-6C show an example of clot size reduction that may be achieved using a thrombectomy device including a spinner, such as that shown in FIG. 3. In the example shown in FIG. 6A, the spinner successfully removed substantially all RBCs from the clot in about three minutes. The appearance of clot before and after spinning, as shown in FIGS. 6B and 6C, respectively, shows the size reduction to less than 10% of the initial volume.

FIG. 7 is an exemplary SEM image of a blood clot showing the clot is mainly composed of RBCs trapped in fibrin fiber networks.

FIGS. 8A and 8B show SEM images of a clot, e.g., the clot in FIGS. 6B and 6C, respectively, showing that the original clot contains >80 vol % of RBC and, after spinning, the white clot is a highly densified fibrin fiber network.

FIG. 9A is a graph showing an example of CFD results on pressure drop in a spinner under different spinning frequencies, demonstrating higher frequency spinning leads to larger pressure drop for better suction.

FIG. 9B is a graph showing examples of centerline pressure profile of a spinner device spinning at various frequencies.

FIG. 9C is a graph showing examples of maximum centerline pressure drop comparison at various blade lengths of a spinner device spinning at various frequencies.

FIGS. 10(1) to 10(10) show examples of CFD results on pressure distribution at the centerline of a milli-spinner (FIG. 10) with different blade sizes during spinning.

FIGS. 11A and 11B show exemplary images from a particle image velocimetry (PIV) system used to evaluate and optimize the suction performance of a milli-spinner. In this example, suction performance of a 2.5 mm spinner is shown with a spinning frequency is 1600 rpm. The arrows denote the fluid velocity field.

FIGS. 12A and 12B show an example of an advancer device that may be provided on a proximal end of a thrombectomy device, such the device shown in FIG. 3.

FIG. 13 is a graph showing experimental efficacy results of a spinner device, under a spinning speed of forty thousand rpm, to reduce 0.03 gram, 0.05 gram, 0.07 gram, and 0.09 gram of formed clots to 30% of initial volume within a tube with water flowing to represent blood flow within a vessel.

FIG. 14 is a graph showing exemplary release rates of a drug carried within a spinner tip depending on rotation speed of the spinner tip.

FIGS. 15A and 15B visually demonstrate exemplary drug release by a spinner tip during experiments at spinner speeds of forty thousand rpm (FIG. 15A) and ten thousand rpm (FIG. 15B) with the intensity of color representing the differences in release rate.

FIG. 16 is a graph comparing localized suction that may be generated by the spinner tips shown in FIGS. 4A-4C.

FIGS. 17A and 17B show an example of an apparatus and system for performing thrombectomy procedures including an aspiration catheter or sheath and a spinner device including a flexible rotation shaft carrying a spinner head movable axially within the catheter, an outer sleeve surrounding the rotation shaft, and a motor coupled to the rotation shaft to rotate the shaft and spinner head.

FIG. 18A shows distal portions of an exemplary spinner device and aspiration catheter that may be included in the system of FIG. 17. As shown, the spinner device includes a wing-stopper on the rotation shaft between the spinner head and sleeve distal end, and the aspiration catheter includes an internal ring to limit distal movement of the spinner device.

FIG. 18B shows the distal portion of the spinner device of FIG. 18A inserted into the aspiration catheter such that the wing-stopper interacts with the internal ring to limit distal movement of the spinner device.

FIG. 19 is a detail of an exemplary internal ring that may be provided on an aspiration catheter, such as that shown in FIGS. 18A and 18B.

FIG. 20 is a detail of an exemplary wing-stopper ring that may be provided on a spinner device, such as that shown in FIGS. 18A and 18B.

FIG. 21A shows an exemplary set of markers that may be provided on a spinner device outer sleeve and aspiration catheter, such as that shown in FIGS. 18A and 18B.

FIG. 21B shows the outer sleeve of FIG. 21A advanced into the aspiration catheter such that the markers are axially aligned with one another.

FIG. 22A shows distal portions of another exemplary spinner device and aspiration catheter that may be included in the system of FIG. 17. As shown, the spinner device includes a wing-stopper on the spinner head, and the aspiration catheter includes an internal ring to limit distal movement of the spinner device.

FIG. 22B shows the distal portion of the spinner device of FIG. 22A inserted into the aspiration catheter such that the wing-stopper interacts with the internal ring to limit distal movement of the spinner device.

FIG. 23 is a detail of exemplary stopper wings that may be provided on a spinner head, such as that shown in FIGS. 22A and 22B.

FIG. 24A shows distal portions of yet another exemplary spinner device and aspiration catheter that may be included in the system of FIG. 17. As shown, the spinner device includes a wing-stopper on the outer sleeve, and the aspiration catheter includes an internal ring to limit distal movement of the spinner device.

FIG. 24B shows the distal portion of the spinner device of FIG. 24A inserted into the aspiration catheter such that the wing-stopper interacts with the internal ring to limit distal movement of the spinner device.

FIG. 25A shows distal portions of still another exemplary spinner device and aspiration catheter that may be included in the system of FIG. 17. As shown, the spinner device includes a wing-stopper and free-floating on the rotation shaft and a blocker fixed on the rotation shaft adjacent the wing-stopper, and the aspiration catheter includes an internal ring to limit distal movement of the spinner device.

FIG. 25B shows the distal portion of the spinner device of FIG. 25A inserted into the aspiration catheter such that the wing-stopper interacts with the internal ring to limit distal movement of the spinner device and the blocker prevents proximal movement of the wing-stopper.

FIG. 26 is a detail of an exemplary blocker ring that may be provided on a rotation shaft, such as that shown in FIGS. 25A and 25B.

FIG. 27A shows an example of a wing-stopper having a uniform axial length that may be provided on a spinner device, such as that shown in FIGS. 25A and 25B.

FIG. 27B shows an example of a modified wing-stopper that may be provided on a spinner device, such as that shown in FIGS. 25A and 25B. The modified wing-stopper includes wings having a tapered proximal shape.

FIG. 27C shows the modified wing-stopper of FIG. 27B free-floating on a rotation shaft adjacent a blocker ring, with the tapered wings configured to minimize contact with the blocker ring.

FIG. 28A shows distal portions of another exemplary spinner device and aspiration catheter that may be included in the system of FIG. 17. As shown, the spinner device includes a wing-stopper proximal to a spinner head and the catheter includes a tubular section attached to its distal end that includes features that limit distal movement of the spinner head.

FIG. 28B shows the distal portion of the spinner device of FIG. 28A inserted into the aspiration catheter such that the wing-stopper interacts with the features on the tubular section to limit distal movement of the spinner device.

FIG. 29A shows distal portions of another exemplary spinner device and aspiration catheter that may be included in the system of FIG. 17. As shown, the catheter includes a secondary lumen within a primary lumen and the spinner device received in the secondary lumen.

FIG. 29B shows the distal portion of the spinner device of FIG. 29A inserted into the secondary lumen of the catheter until the spinner head is positioned adjacent an outlet of the catheter.

FIG. 30A shows distal portions of another exemplary spinner device and aspiration catheter that may be included in the system of FIG. 17. As shown, an outer sleeve of the spinner device includes a plurality of balloons spaced apart axially from one another configured to expand and contact the inner wall of the catheter to center the spinner head along a centerline of the catheter.

FIG. 30B shows the distal portion of the spinner device of FIG. 30A inserted into the catheter with the balloons inflated to center the spinner head.

FIG. 30C is a cross-sectional detail showing the balloons of FIGS. 30A and 30B expanded within the catheter lumen to center the spinner device.

FIG. 31A shows distal portions of another exemplary spinner device and aspiration catheter that may be included in the system of FIG. 17. As shown, the catheter includes a mesh across its outlet that prevent the spinner head from advancing from the outlet.

FIG. 31B shows the distal portion of the spinner device of FIG. 31A inserted into the aspiration catheter until the spinner head contacts the mesh to prevent further distal movement.

FIG. 32A shows distal portions of another exemplary spinner device and aspiration catheter that may be included in the system of FIG. 17. As shown, the catheter and spinner device include a set of magnets configured attract one another to hold the spinner head stationary adjacent the outlet of the catheter.

FIG. 32B shows the distal portion of the spinner device of FIG. 32A inserted into the aspiration catheter until the magnets are aligned to limit axial movement of the spinner head.

FIG. 33A shows distal portions of another exemplary spinner device and aspiration catheter that may be included in the system of FIG. 17. As shown, the catheter includes an internal ring and the spinner head includes two sets of wings spaced apart axially from one another that interact with the internal ring to axially constrain the spinner head.

FIG. 33B shows the distal portion of the spinner device of FIG. 33A inserted into the aspiration catheter until the wings interact with the internal ring to limit axial movement of the spinner head.

FIG. 34A shows distal portions of another exemplary spinner device and aspiration catheter that may be included in the system of FIG. 17. As shown, the catheter includes two internal rings spaced apart from one another and the spinner device includes a set of wings that can be received between the rings to axially constrain the spinner head.

FIG. 34B shows the distal portion of the spinner device of FIG. 34A inserted into the aspiration catheter such that the wing-stopper positioned between the internal rings to limit axial movement of the spinner head.

FIG. 35A shows distal portions of another exemplary spinner device and aspiration catheter that may be included in the system of FIG. 17. As shown, the catheter includes two sets of bumps spaced apart axially from one another and the spinner device includes a ring supported concentrically around the spinner device that can be received between the bumps to axially constrain the spinner head.

FIG. 35B shows the distal portion of the spinner device of FIG. 38A inserted into the aspiration catheter such that the wing-stopper positioned between the bumps to limit axial movement of the spinner head.

FIGS. 36A-36D are details showing fibrin residue from a clot being entangled on a spinner head to mechanically capture the clot and allow leftover clot to be retracted into a catheter when the spinner head is retracted.

FIGS. 37A and 37B are details of a shredding tip that may be provided on a spinner head such that the tip may be rotated after engaging a clot to break the clot into pieces and aspirated into a catheter.

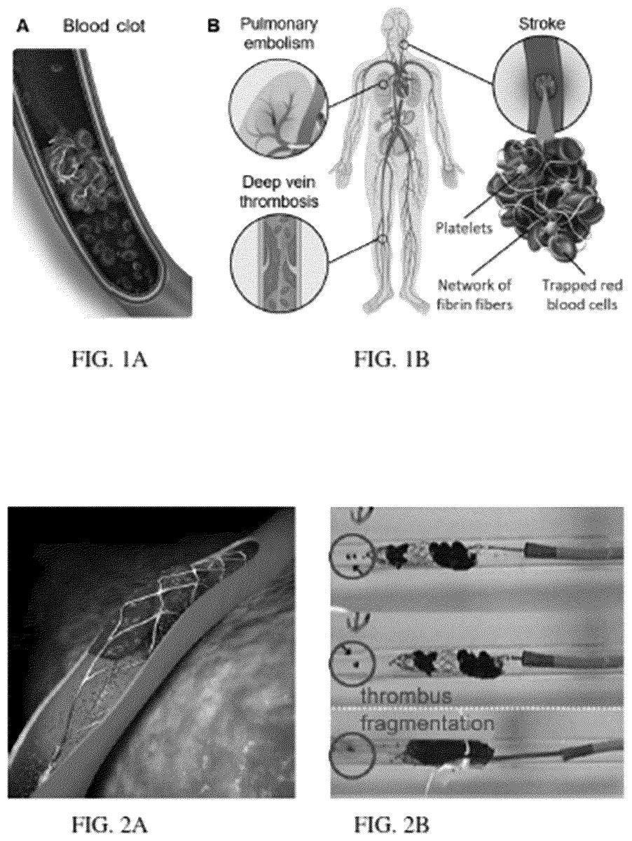

The drawings are not intended to be limiting in any way, and it is contemplated that various examples of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

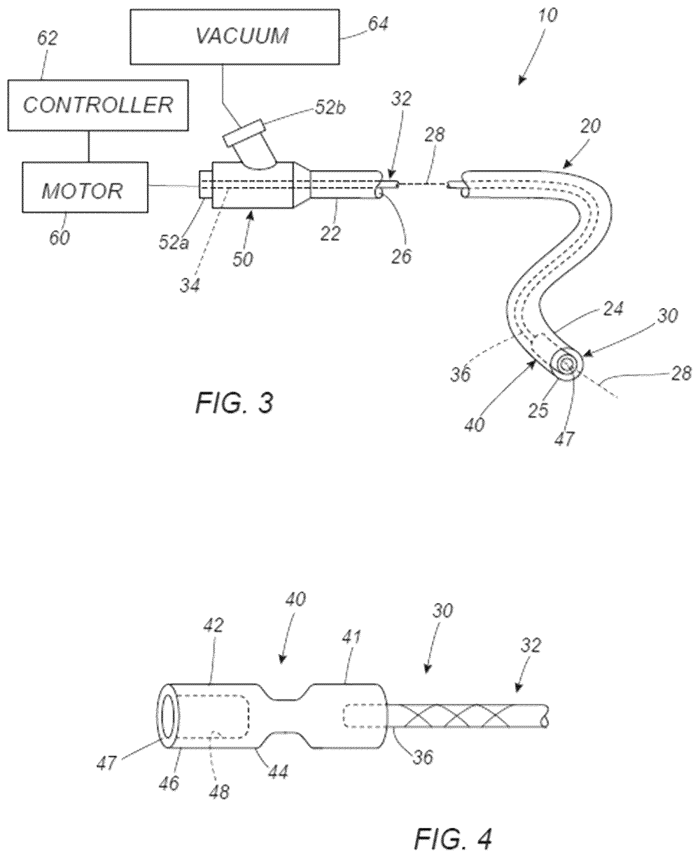

Turning to the drawings, FIG. 3 shows an example of a thrombectomy device or apparatus 10 that may be used with the systems and methods described herein throughout. In general, apparatus 10 includes an outer catheter, sheath, sleeve, or other member 20, and a spinner device 30 including a spinner tip or member 40 coupled to a shaft 32 that may be introduced through or otherwise positioned in the catheter 20. Optionally, the device 10 may include a sleeve (not shown in FIG. 3, sec, e.g., sleeve 121 shown in FIGS. 12A and 12B), which may be positioned and/or otherwise provided around the shaft 32. The sleeve may help protect the inner surface of the catheter 20 when the shaft 32 rotates at high speeds, may reduce vibration resulting from the shaft 32 and/or spinner device 30 rotation, and/or may facilitate centering and/or stabilizing the spinner device 30.

Generally, as shown, the catheter 20 is an elongate tubular member including a proximal end 22 including a handle or hub 50, a distal end 24 sized for introduction into a blood vessel or other body lumen, and one or more lumens 26 extending between the proximal and distal ends 22, 24, e.g., along a longitudinal axis 28. For example, as shown, a main lumen 26 may be provided that communicates with one or more ports 52 in the handle 50 and extends to an outlet 25 in the distal end 24. Optionally, the catheter 20 may include one or more additional lumens extending at least partially between the proximal and distal ends 22, 24, e.g., a guidewire lumen for receiving a guidewire or other rail, a steering element lumen, and the like (not shown). It should also be understood that while catheter 20 is shown here as being tubular, it need not have a perfect circular cross-section. Indeed, it may be partially tubular, or have any other suitable geometry. Similarly, when reference is made to a lumen, it should be understood that a lumen may be a partial lumen, groove, or slit.

The catheter 20 may be constructed using conventional biocompatible materials and/or methods, e.g., formed from plastic, various polymers, metal, composite materials, having a substantially homogenous construction between the proximal and distal ends 22, 24. Alternatively, the construction may vary along the length of the catheter 20 to provide desired properties, e.g., to provide a proximal portion that is substantially rigid or semi-rigid, e.g., providing sufficient column strength to allow the distal end 24 of the catheter 20 to be pushed or otherwise manipulated from the proximal end 22, while a distal portion adjacent the distal end 24 may be substantially flexible to facilitate advancement through tortuous anatomy. In either construction, the catheter 20 may also be coated or layered (e.g., with a lubricious material) to aid in advancement.

The shaft 32 of the spinner device 30 may be an elongated flexible member including a proximal end 34 and a distal end 36 sized to be received within the lumen 26 of the catheter 20. The shaft 32 may be sufficiently long to allow introduction of the distal end 36 into a target blood vessel, e.g., through or along with the catheter 20, while the proximal end 34 remains outside the patient's body. For example, the shaft 32 may be a solid or tubular cable, e.g., including a plurality of helically wound inelastic fibers, wires, and the like, constructed to translate rotation from the proximal end 34 to the distal end 36 to rotate the spinner tip 40, e.g., with sufficient torsional strength such that rotation of the proximal end 34 causes directly corresponding rotation of the distal end 36, and consequently the spinner tip 40 at relatively high speeds, when coupled to a motor 60 and/or controller 62, as described further elsewhere herein as described elsewhere herein.

Optionally, a sleeve or other tubular member (not shown) may be provided around the shaft 32, e.g., to prevent the shaft 32 from contacting the inner wall of the catheter 20 when introduced into the lumen 26 and rotated by the motor. The sleeve may be formed from lubricious material, e.g., PTFE, and/or may include a coating on an inner surface thereof to reduce friction and/or otherwise facilitate the shaft 32 rotating in the sleeve. The sleeve may be axially fixed relative to the shaft 32, e.g., such that the sleeve extends from the proximal end 34 to the distal end 36 immediately proximal to the spinner tip 40. Alternatively, the sleeve may be separate from the spinner device 30, e.g., such that the sleeve may be introduced into the lumen 26 of the catheter 20 before introducing the spinner tip 40 and shaft 32.

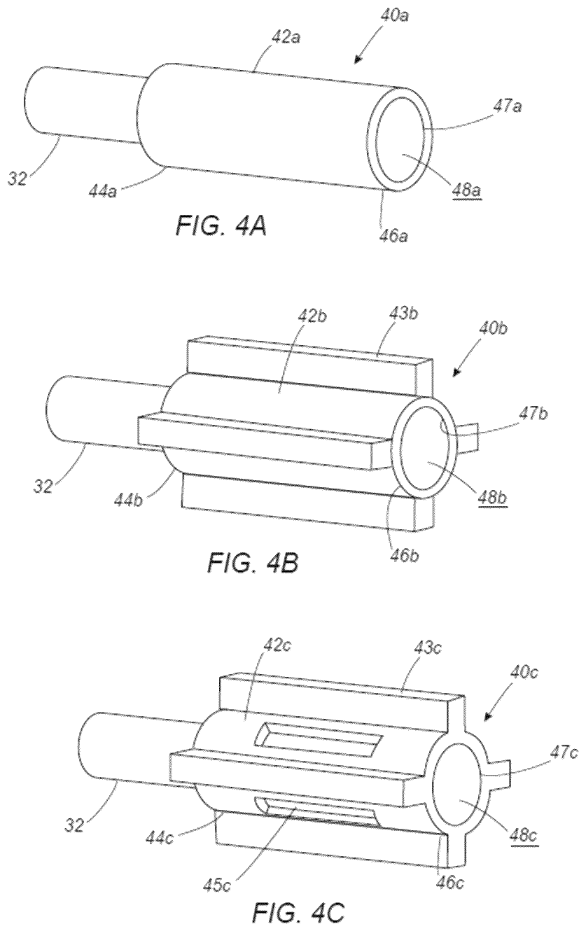

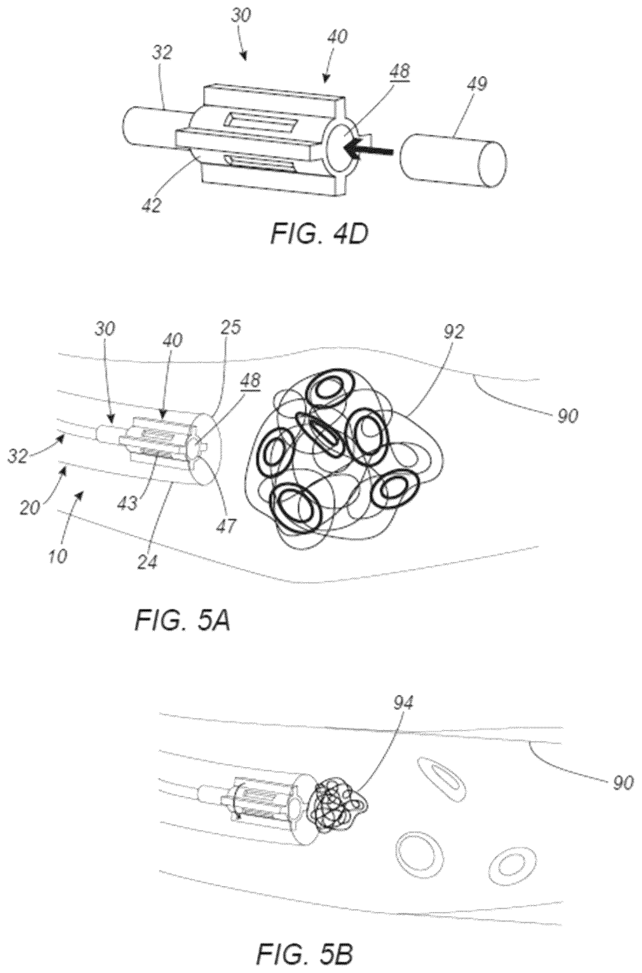

Generally, as shown in FIG. 4A, the spinner tip 40 may include a cylindrical or other annular body 42 including a closed proximal end 44 that may be connected to the shaft 32, and an open distal end 46 including an inlet 47 communicating with an interior cavity 48 of the body 42. Optionally, a hub 41 may be provided on the proximal end 44, which may facilitate attaching the spinner tip 40 to the distal end 36 of the shaft 32. For example, the hub 41 may be used to substantially permanently attached the tip 40 to the shaft 32, e.g., by one or more of over-molding, fusing, sonic welding, cooperating connectors, and the like. For example, as shown, the hub 41 may include a recess that may receive the distal end 36 of the shaft 32, and the hub 41 may be bonded, melted, press-fit, and/or otherwise permanently attached over the shaft 32.

Optionally, the spinner device 30 may include one or more markers, e.g., radiopaque rings or deposited material, at desired locations, e.g., on the distal end 36 of the shaft 32 and/or on the spinner tip 40, which may facilitate monitoring introduction and/or operation of the device 30 during a procedure, e.g., using fluoroscopy, Xray, ultrasound, or other external imaging. In addition or alternatively, the shaft 32 may be constructed from radiopaque materials, which may facilitate monitoring the shaft 32 during introduction and/or manipulation during a procedure.

Turning to FIGS. 4A-4C, examples of spinner tips 40 are shown that may be used with the devices, systems and methods provided herein, e.g., on the distal end 36 of the shaft 32. For example, in FIG. 4A, the spinner tip 40a includes a cylindrical body 42a that includes a substantially uniform annular wall extending between the proximal end 44a and the distal end 46a, which includes inlet 47a communicating with the interior cavity 48a. The body 42a may have a substantially flat, e.g., atraumatic distal face, which may facilitate placement against clot without macerating the clot when the body 42a is rotated. Optionally, as shown in FIG. 4B, the tip 40b may include one or more blades, struts, or other exterior features on the cylindrical body 42b to enhance localized suction and/or shear force on the clot, e.g., a plurality of elongate blades 43b extending at least partially between the proximal and distal ends 44b, 46b of the cylindrical body 42b. As shown, four blades 43b are shown that are spaced evenly around the cylindrical body 42b, e.g., offset about ninety degrees from one another around the circumference of the wall of the body 42b. Also as shown, the blades 43b may extend the length of the cylindrical body 42b or may only extend partially between the proximal and distal ends 44b, 46b. It should be understood that while equal spacing is shown here, unequal spacing is also contemplated. In addition, while four blades are shown, any number of blades may be used, e.g., two, three, four, five, six, seven, etc. Similarly, it should be understood that the blades need not be of equal dimension (e.g., length, width, height). In some variations, the blades may be of unequal dimensions.

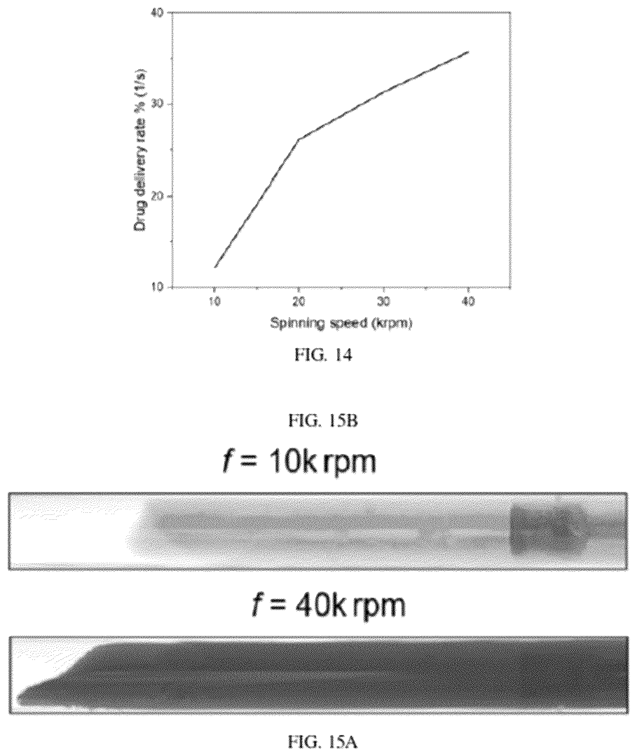

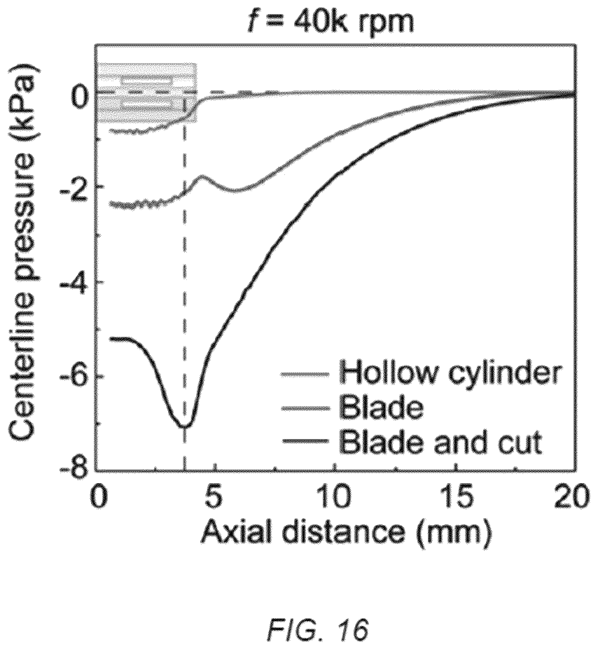

In addition or alternatively, as shown in FIG. 4C, the tip 40c may include one or more slits or other openings in the wall of the cylindrical body 42c, e.g., extending radially outwardly from the cavity 48c through the wall to the outer surface of the annular body 42c. For example, as shown, an elongate slit 45c is provided between each adjacent blade 43c, the slit 45c extending partially between the proximal and distal ends 44c, 46c of the cylindrical body 42c. The slits 45c may enhance the localized suction within the cavity 48c and inlet 47c, as described elsewhere herein. Alternatively, slits may be provided only between some of the blades and/or a plurality of slits may be provided in a cylindrical body without blades (not shown), as desired. Although four slits and blades are shown in these examples, it will be appreciated that any desired number of slits and/or blades may be provided on the spinner tip spaced around the annular body, e.g., two, three, four, or more. Similarly, it should be appreciated that the slits need not be of equal size. Adding blades and adding both blades and slits to the spinner tip may substantially increase the localized suction that is generated by the spinner tips 40a-40c shown in FIGS. 4A-4C. For example, FIG. 16 shows experimental results comparing localized suction generated at 40,000 rom for spinner tips without blades (hollow cylinder only, similar to 40a) with spinner tips including blades (similar to 40b) and including both blades and slits (similar to 40c).

The various dimensions of the spinner tip 40 may be sized to be inserted into a target blood vessel and/or to generate desired localized suction pressures, as described further elsewhere herein. In some examples, the body 42 may have a length between about one to five millimeters (1.0-5.0 mm), e.g., about 4.2 mm, and a wall thickness between about 0.05-0.15 mm, e.g., about 0.09 mm or 0.15 mm; the inlet 47 may have a diameter between about 0.5-1.2 mm, e.g., about 0.72 mm or 1.2 mm. The blades 43 may have heights between about 0.1-0.7 mm, e.g., about 0.31 mm or 0.51 mm, and widths between about 0.1-0.5 mm, e.g., about 0.24 mm or 0.40 mm. The slits 45 may have lengths between about 0.5-2.5 mm, e.g., about 2.1 mm, and widths between about 0.2-0.8 mm around the radius, e.g., about 0.45 mm or 0.75 mm.

In another alternative, the spinner tip may include a plurality of helical blades (not shown), e.g., extending at least partially around the circumference of the annular body and/or along the length of the annular body. Optionally, in this alternative, one or more helical slits or other openings may be provided between one or more of the helical blades. It will be appreciated that one or more additional features may be provided on the outer surface of the annular body, in addition to or instead of the blades and/or slits, that may enhance the localized suction generated by the spinner tip.

Returning to FIG. 4, the spinner tip 40 (or any other examples described herein) may be integrally formed as a single piece, e.g., from plastic, metal, composite material, and the like, e.g., manufactured using one or more of 3D-printing, by molding, micro-injection molding, casting, machining, and the like. Alternatively, the annular body 42 and/or hub 41 may be formed from one or more substantially continuous processes, such as extrusion and the like, e.g., to allow manufacturing a single assembly that may be separated into individual tips. Optionally, the annular body may be formed without the slits and/or blades, which may be added or formed during subsequent processing of the annular body, if desired.

The spinner tip 40 may be substantially rigid or, alternatively, the material of the spinner tip 40 may be flexible or semi-rigid, e.g., formed from relatively soft material, such as elastomeric material, e.g., silicone, or soft plastics, such that the distal end 46 of the annular body 42 provides a substantially atraumatic tip that may minimize risk of damaging tissue contacted by the tip. For example, the spinner tip 40 may be formed from relatively soft material, such as an elastomeric material with stiffness less than 40 MPa. Thus, the spinner tip 40 may be able to recover from deformation and retain its shape, e.g., by bending less than about one hundred eighty degrees (180)° degrees and/or twisting less than about five hundred forty degrees (540°).

Similarly, the blades 43 may be formed from flexible and/or soft material and/or may include rounded or other atraumatic outer edges, e.g., to prevent damaging the vessel wall and/or causing the clot to be macerated when the spinner tip 40 is rotated.

The spinner tip 40 may have a diameter or other outer cross-section sized to be received within the lumen 26 of the catheter 20 while allowing the tip 40 to rotate freely, e.g., providing clearance around the tip 40 within the lumen. For example, the tip 40 may have an outer diameter between about 1.2-2.5 millimeters, e.g., having an outer diameter of about two millimeters (2 mm) or less.

Optionally, as shown in FIG. 4D, the spinner tip 40 may include a drug delivery member 49, e.g., for delivering one or more therapeutic and/or diagnostic agents. For example, the drug delivery member 49 may include a cylindrical body sized to be received in the cavity 48 of the spinner tip 40. In one example, the drug delivery member 49 may be formed from porous material, e.g., such that one or more agents may be loaded into the porous material and released, e.g., when the spinner tip 40 is rotated within a blood vessel. In addition or alternatively, the drug delivery member 49 may be formed from bioabsorbable or dissolvable material, e.g., to release the one or more agents as the material dissolves or otherwise breaks down.

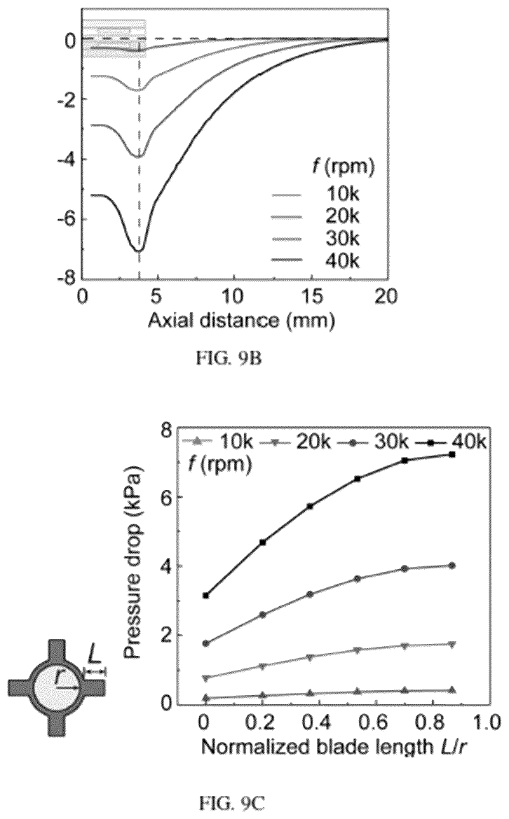

Returning to FIG. 3, the spinner tip 40 is connected to the shaft 32, which is coupled, in turn, to a motor 60 that provides the torque to rotate the spinner tip 40. For example, the proximal end 34 of the shaft 32 may include a connector (not shown) to couple the shaft 32 to the motor 60, e.g., via an external drive shaft, cable, and the like (also not shown). The motor 60 may be configured to rotate the spinner tip 40 at desired speeds, e.g., at least 1000 rpm, or at least 10,000 rpm, e.g., between about 1,000 and 200,000 rpm, between about 4,000 and 50,000 rpm, between about 10,000 and 40,000 rpm, between about 20,000 and 40,000 rpm, or between about 30,000 and 40,000 rpm, e.g., around 10,000 rpm or around 40,000 rpm, which may generate a desired localized suction pressure at the inlet 47 of the tip 40. For example, with the spinner tip rotating at 40,000 rpm, localized suction of about seven thousand Pascals (7kPa) may be generated, while at 10,000 rpm, five hundred Pascals (0.5 kPa) may be generated, e.g., as shown in FIGS. 9A-9C.

In one example, the motor 60 may be configured to rotate the spinner tip 40 at a single set speed. Alternatively, the speed of the motor 60 may be variable, e.g., manually using an actuator of the controller 62 coupled to the motor 60, which may be adjusted by the user to modify the rotation speed of the spinner tip 40. Alternatively, the controller 62 may be configured to initially operate the motor 60 at a relatively lower speed and then the speed may be automatically increased to rotate the spinner tip 40 at a desired active speed. For example, the initial speed may be used to mechanically engage the clot, and then the speed may be increased, e.g., to rapidly remove red blood cells from the clot and reduce clot size, as described elsewhere herein.

Different from existing aspiration thrombectomy devices, which require continuous extraction of blood from the vessel being treated, the spinner devices herein may generate a highly localized suction force without removing any fluid from the vessel. Optionally, the devices may be used in conjunction with aspiration, e.g., by connecting a source of vacuum 64 to the catheter 20 or introducing a separate suction device (not shown), as described further elsewhere herein.

A controller 62 may be coupled to the motor 60 to control operation of the device 10, e.g., to allow a user to turn the motor 60 off and on to rotate the spinner tip 40 and generate the localized suction with a blood vessel. Optionally, the controller 62 may include one or more actuators, e.g., switches and the like (not shown), to activate/deactivate the motor and/or to adjust the speed, if desired. In addition or alternatively, the controller 62 may include an actuator (also not shown) to advance and/or retract the shaft 32 axially, e.g., relative to the catheter 20, e.g., using an advancer device 150, such as that shown in FIGS. 12A and 12B and described elsewhere herein. In another option, the controller 62 may include a robotic control system to control axial movement of the shaft 32 remotely, if desired.

In one example, the spinner device 30 and catheter 20 are assembled together, e.g., such that the catheter 20 and spinner device 30 are introduced together into a patient's body, e.g., similar to the device 150 shown in FIGS. 12A and 12B. In this way, the devices described herein are part of a pre-assembled system or kit. Alternatively, the spinner device 30 may be separate from the catheter 20, i.e., including the shaft 32 and spinner tip 40, e.g., such that the catheter 20 may be introduced initially into a patient's body, and, once the distal end 24 and outlet 25 are positioned adjacent a target clot, the spinner device 30 may be introduced into the catheter 20 and advanced to position the spinner tip 40 adjacent the outlet 25. In this way, the devices described herein may be assembled just prior to, or during, use. In this example, the handle 50 of the catheter 20 may include a port 52a that allows the spinner device 30 to be inserted into and removed from the lumen 26 of the catheter 20, which may include one or more hemostatic seals, e.g., to prevent fluid from leaking from the port 52a while allowing the shaft 32 of the spinner device 30 to be advanced through the port 52a into the lumen 26. Alternatively, the spinner device 30 may be permanently integrated with the catheter 20, e.g., such that the spinner device 30 cannot be removed but may be advanced and/or retracted axially within the lumen 26, e.g., using the advancer device 150.

Optionally, the catheter 20 and/or shaft 32 may include one or more stops or other safety features (not shown) to limit axial movement of the shaft 32 and thus help prevent accidental shearing of non-clot tissue during use. For example, a stop may be provided within the handle 50 that prevents the spinner device 30 from being advanced to expose the spinner tip 40 from the outlet 25 of the catheter 20. Alternatively, the stop(s) may allow the spinner tip 40 to be exposed partially or entirely from the outlet 25, if desired. Optionally, another stop may be provided that allows the spinner tip 40 to be retracted a desired distance proximally from the outlet 25, e.g., to allow residual fiber from a reduced or dissolved clot to be aspirated or otherwise directed into the outlet 25, as described further elsewhere herein. In another alternative, the spinner device 30 may be axially fixed relative to the catheter 20, e.g., such that the spinner tip 40 is located within the lumen 26 with the distal end 46 of the spinner tip 40 located immediately adjacent the outlet 25.

If the spinner device 30 is permanently integrated with the catheter 20, the proximal end 34 of the shaft 32 may extend from the port 52a on the handle 50 (whether the shaft 32 is movable axially or axially fixed). The proximal end 34 of the shaft 32 may include a connector configured to couple the shaft 32 to a driveshaft of the motor 60 (not shown), e.g., to allow the thrombectomy device 10 to be connected and disconnected from the motor 60. In this example, the thrombectomy device 10 may be a single-use, integral device that may be provided to a user for use during a procedure, after which the device 10 may be discarded. Alternatively, if the spinner device 30 is provided separately from the catheter 20, both may be single-use and/or disposable or one or both may be reusable, e.g., after cleaning and/or sterilization. In a further alternative, the spinner device 30 may be provided and/or introduced into a patient's body without the catheter 20, if desired.

Optionally, as shown in FIGS. 12A and 12B, an advancer device 150 may be provided on the proximal end of a thrombectomy device, e.g., in place of the handle 50 shown on the device 10 of FIG. 3. Generally, the advancer device 150 includes a stationary handle portion 152 coupled to a proximal end 122 of a catheter 120, and a slider portion 154 coupled to a shaft 132 carrying a spinner tip on its distal end (not shown). The catheter 120, shaft 132, and spinner tip may be generally constructed similar to any of the other devices herein, similar to the device 10 shown in FIG. 3. Optionally, a sleeve 121 may be provided around the shaft 132 that extends through the catheter 120 to protect the inner surface of the catheter 120 when the shaft 132 rotates, similar to other devices herein.

The components of the advancer device 150 may be provided within an outer housing, e.g., including clamshells or other portions that may be attached together (not shown), e.g., to protect the internal components. Optionally, the outer surface of the housing may be contoured to provide a grip to facilitate holding and/or manipulating the device, or the housing may include a base or other structure for stabilizing the housing relative to a patient during a procedure. Optionally, as shown in FIG. 12B, the advancer device 150 may include a motor 160 and/or a battery 162 or other power source, e.g., for driving the shaft 132 to rotate the spinner tip, similar to other devices herein.

As shown, the slider 154 may be slidably received in a track or other guide in the stationary portion 152, e.g., such that the slider 154 may be directed axially between a first or proximal position and a second or distal position, e.g., to advance and retract the spinner tip relative to the distal end of the catheter 120 (not shown), similar to other devices herein. As shown, a screw 156 or other actuator may be coupled to the slider 154, e.g., to allow an operator to manually direct the slider 154 between the first and second positions. Optionally, the screw 156 may include a fastener that may actuated to secure the slider 154 at a desired position, e.g., to fix the spinner tip relative to the distal end of the catheter 120, e.g., once deployed during a procedure. While a slider is shown here, any suitable actuation member and/or methods may be used, e.g., button, knob, and/or combinations thereof.

During use, with the spinner tip retracted within the catheter 120, the distal end of the catheter 120 may be introduced into a patient's body and advanced to a target location, e.g., adjacent a clot within the patient's vasculature (not shown). Once positioned, the screw 156 may be actuated to advance the spinner tip relative to the catheter 120, e.g., to place the distal face of the spinner tip against or immediately adjacent the clot, similar to other devices herein. The screw 156 may be used to lock the spinner tip's relative position in the catheter 120 once advanced to a desired location. The motor 160 may then be activated to rotate the spinner tip to dissolve and/or reduce the clot, similar to other devices herein. Once the clot is treated, the screw 156 may be actuated to retract the spinner tip back into the catheter 120, and the device may be removed (or directed to one or more additional locations to treat additional clot).

Any of the devices, systems, and methods described herein may optionally include a vacuum source. For example, as shown in FIG. 3, the device 10 may include a source of vacuum 64, e.g., a syringe, suction line, and the like (not shown), that may be coupled to the proximal end 22 of the catheter, e.g., to port 52b on the handle 50 for aspirating material into the lumen. For example, the port 52b may include a Luer fitting or other connectors that allow tubing from the source of vacuum 64 to be removably connected to the port 52b. Before or during advancing and/or activating the spinner tip 40 to reduce or dissolve a clot, the vacuum source 64 may be activated (or may be activated immediately upon advancing the spinner tip 40, if desired) to aspirate dissolved fibrin or other remaining clot material, e.g., into the lumen 26 of the catheter 20, as described further elsewhere herein. The spinner tip 40 may also help reorient and/or reposition the clot relative to the catheter 20, e.g., to enhance contact between the clot and the outlet 25, which may enhance vacuum suction from the lumen 26.

In addition or alternatively, a source of fluid, e.g., a syringe of saline, contrast, and the like, may be connected to the port 52b (or to a separate dedicated port, not shown, if desired). Thus, during use of the catheter 20, the lumen 26 may be flushed and/or the fluid may be delivered through the outlet 25, if desired.

Optionally, the thrombectomy device 10 and/or spinner device 30 may be included in a system or kit including one or more additional devices for use during a thrombectomy procedure. For example, the system may include an occlusion device and/or a capture member (not shown) to prevent fragments of a clot being treated from migrating elsewhere within the patient's vasculature. For example, such devices may be introduced and deployed from the catheter 20, e.g., through the lumen 26 or a secondary lumen. Alternatively, such devices may be introduced and deployed independently of the spinner device, e.g., via a separate catheter, sheath, or other device (not shown) downstream from the target clot.

Suitable additional devices for use within a system or kit as described herein may include, for example, a device carrying a balloon or other expandable member may be provided that may be introduced into a body lumen spaced apart from and/or adjacent the spinner tip 40 and/or catheter outlet 25, and the expandable member may be expanded to at least partially occlude the body lumen to prevent material from the clot migrating from the body lumen. Alternatively, a capture member may be provided for introduction into a body lumen adjacent the spinner tip, e.g., to capture residual fibrin material from the clot being treated by the spinner tip. Such a capture member may include a filter, a snare, a cage, and the like.

The devices and systems herein may be used during a thrombectomy procedure. For example, the spinner device 30 may be introduced into a body lumen, e.g., a vein, artery, and the like, adjacent a target blood clot, and the spinner tip 40 may be rotated to generate localized suction, e.g., to generate localized hydrodynamic forces combining compression and shear forces, to dissolve the clot, reduce clot size, and/or prevent fragmentation of a clot within the body lumen. For example, the spinner device may be deployed to reduce or otherwise dissolve a clot, e.g., by separating the red blood cells from the fibrin and/or other fibrous material. The red blood cells may simply be released within the vessel, e.g., such that the cells are metabolized by the body, and the fibrin may be captured, e.g., directly by the cavity of the spinner device, or using aspiration, a capture device, and the like, and/or treated with a thrombolytic drug or other agent to breakdown, dissolve, and/or otherwise neutralize the residual material before the spinner device is removed from the patient's vasculature.

In an exemplary method, shown in FIGS. 5A and 5B, the distal end 24 of the catheter 20 may be initially introduced into the patient's body, e.g., over a guidewire or other rail (not shown) positioned within the patient's vasculature from a percutaneous access site. The distal end 24 may be advanced to position the outlet 25 within a blood vessel 90 adjacent a target clot 92, as shown in FIG. 5A.

The spinner tip 40 may be introduced into the lumen 26 from the proximal end of the catheter 20 and advanced to position the inlet 47 adjacent the outlet 25 of the catheter 20 and, consequently, adjacent the clot 92, as shown in FIG. 5A. Alternatively, as described elsewhere herein, the spinner device 30 may be provided within the catheter 20, e.g., such that the spinner tip 40 is positioned adjacent the clot 92 at the same time as the catheter 20 is introduced. Optionally, a sleeve (not shown) may be provided around the shaft 32 within the lumen 26, which may be introduced with the spinner device 30 or may be introduced into the lumen 26 before inserting the spinner device 30.

Optionally, manipulation of the catheter 20 and/or spinner device 30 may be monitored using external imaging, e.g., fluoroscopy, Xray, ultrasound, and the like. For example, as described elsewhere herein, the spinner device 30 may include one or more radiopaque markers, e.g., on the distal end 36 of the shaft 32 and/or spinner tip 40, and/or the shaft 32 may be formed from radiopaque material that may be monitored to facilitate positioning the spinner tip 40 adjacent the clot 92. Optionally, contrast may be introduced into the blood vessel 90, e.g., via the port 52b, a separate port on the proximal end 22 of the catheter 20, or a separate device (not shown), to facilitate locating the clot 92 and positioning the spinner tip 40, which may be introduced through the catheter 20 (e.g., through the main lumen 26 or a separate lumen) or through a separate device. In addition or alternatively, the catheter 20 and/or spinner device 30 may be monitored using intravascular imaging systems and methods, such as intravascular ultrasound imaging, optical coherence tomography, and the like.

Once the device is positioned as desired relative to the clot 92, the spinner tip 40 may then be rotated, e.g., by activating the motor 60 (shown in FIG. 3), to generate localized suction within the inlet 47, thereby drawing the clot 92 against the distal end 24 of the catheter 20, e.g., to press the clot 92 against the distal end 46 of the spinner tip 40. As described above, the speed of the spinner tip 40 may be fixed or adjustable, e.g., manually by the user and/or automatically by the controller 60. For example, the spinner tip 40 may positioned against or immediately adjacent the clot 92 and rotated to generate shear forces that separate red blood cells from the clot. In one method, only the distal face of the spinner tip 40 is placed in contact with the clot and the spinner tip 40 is not rotated inside the clot 92, e.g., to prevent maceration.

Unlike other thrombectomy devices that attempt to catch or macerate the entire clot (leading to fragmentation of the clot into pieces), the devices and systems of the present disclosure help separate the red blood cells of the clot from the complex fibrin network, thus leading to greater efficiency and efficacy. That is, the spinning motion of the spinner tip 40 may generate a shear force which, in combination with the compression from the suction to squeeze out red blood cells trapped in the clot 92, which may escape through the slits 43 and/or otherwise from the cavity 48 of the spinner tip 40 into the vessel. Thus, unlike macerator devices, which mechanical break the clot into pieces and risk fragments traveling to other locations in the patient's body, the devices herein allow the red blood cells to be removed to reduce the volume of the clot without breaking up the residual fibrin material. The residual material may remain substantially intact and then removed, as described elsewhere herein. Further, macerator devices may be incapable of breaking up rich and/stiff fibrin networks of some clots and so may be incapable of removing such clots, while the devices herein allow such residual fibrin network to be captured and/or otherwise removed regardless of the stiffness of the residual material.

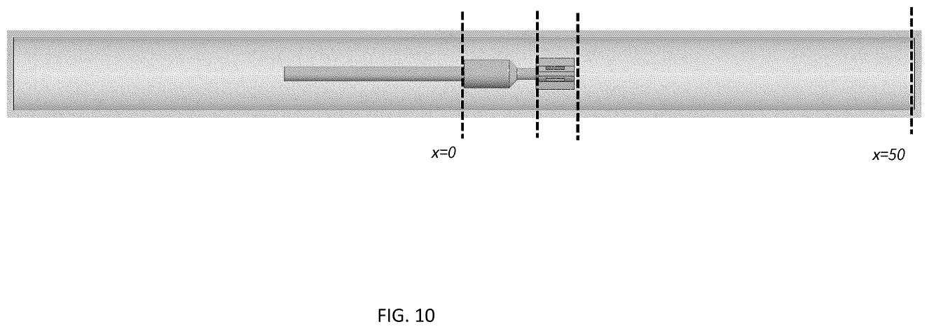

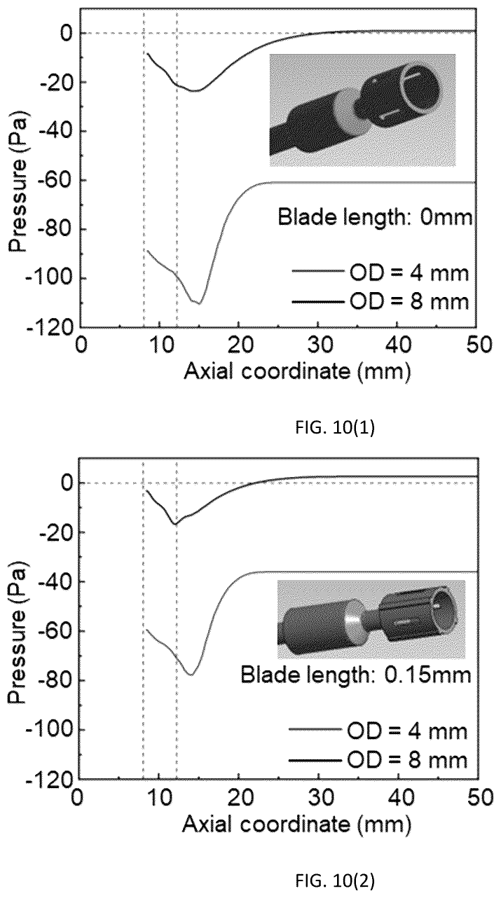

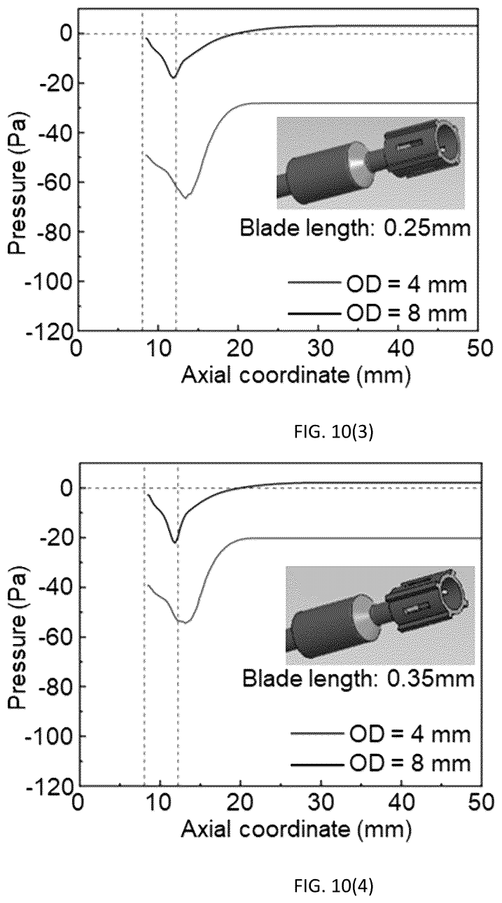

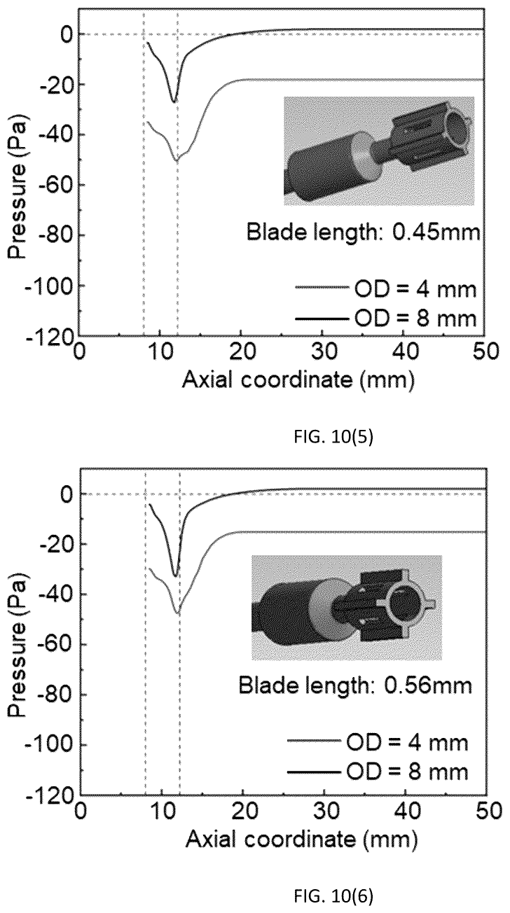

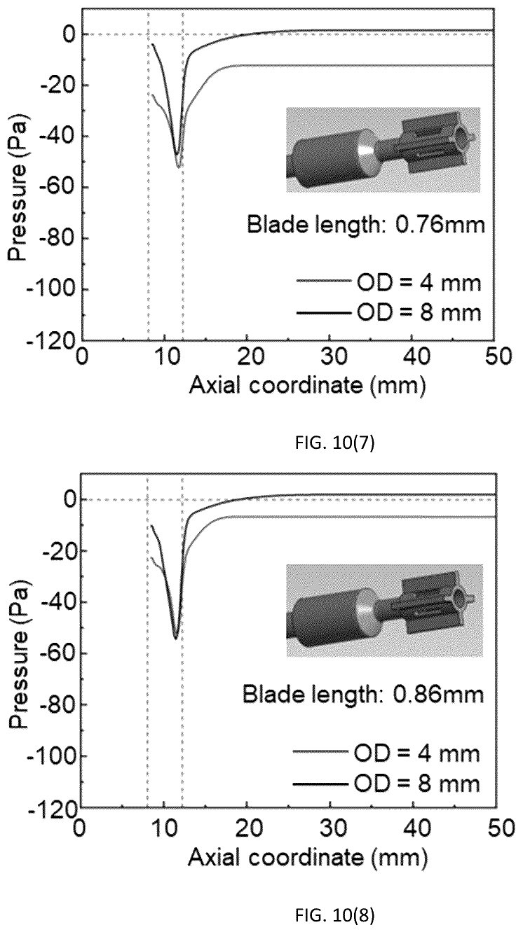

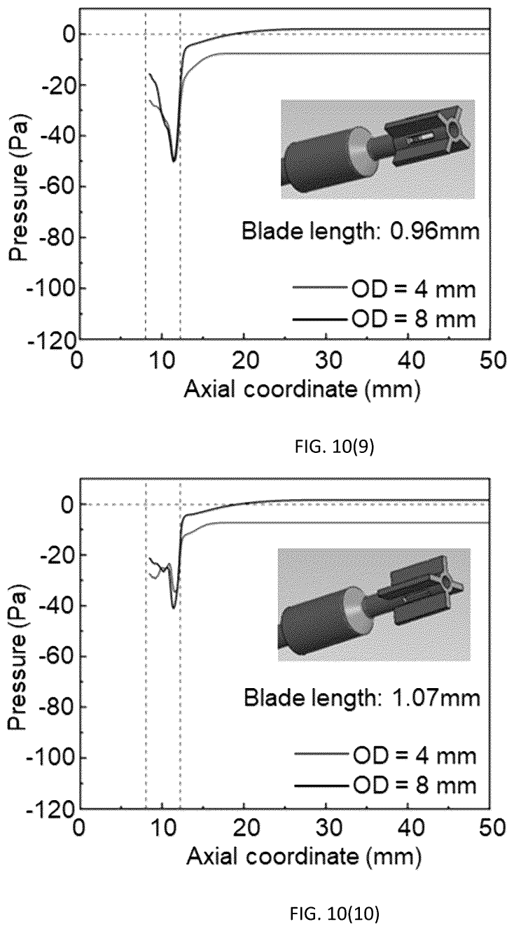

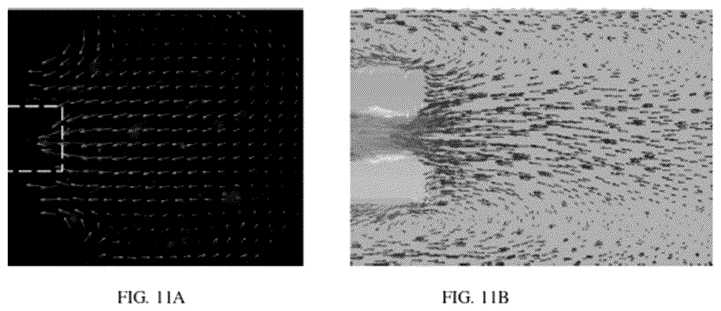

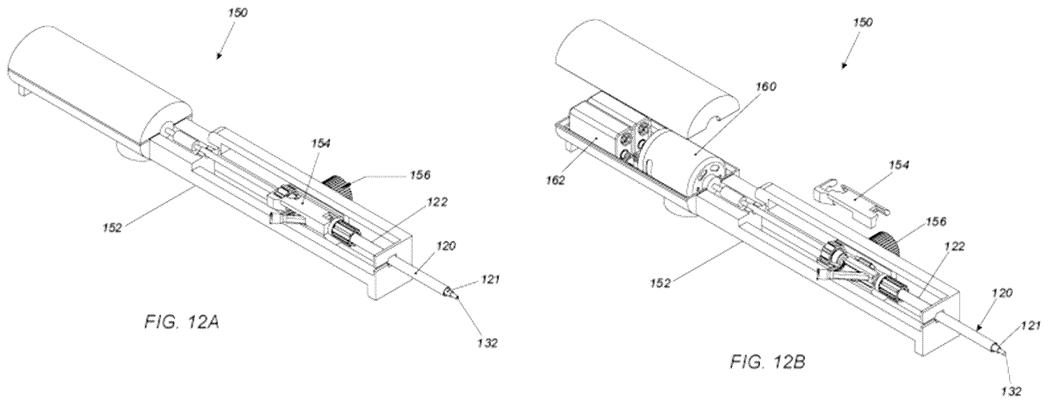

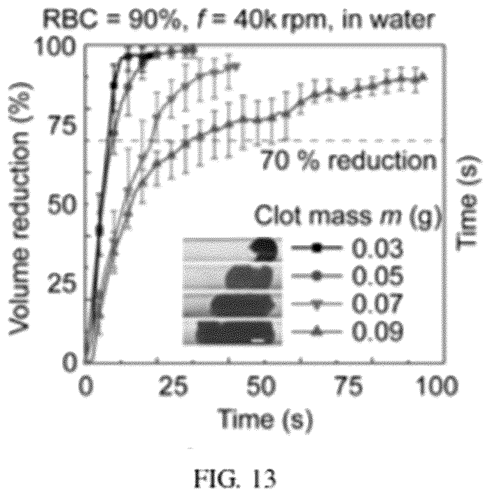

For example, as shown in FIGS. 9A-9C, increased spinning speed may lead to higher localized suction. The magnitude of suction is positively related to clot-dissolving efficacy as shown in FIG. 13. As shown in FIG. 13, the reduction effect is expressed as the time it takes for a clot to reach certain volume reduction amount (in this experiment, 70% volume reduction was the chosen as the evaluation standard). At 40,000 rpm, no breakup of the thrombus has been observed. At even higher spinning speeds, clot-dissolving efficiency is expected to increase. FIGS. 11A and 11B show exemplary pressures and localized flow that may be generated when the spinner tip 40 is rotated at various speeds, e.g., such as those shown in FIGS. 9A-9C and 10.

In this way, the spinner tip 40 may rapidly separate the red blood cells, e.g., in less than two minutes, leaving a compacted fibrin fiber network 94, e.g., as shown in FIG. 5B. Optionally, additional vacuum may be applied, e.g., by connecting a source of vacuum 64 to the proximal end 22 of the catheter 20, as shown in FIG. 3, to generate substantially continuous suction through the outlet 25 into the lumen 26. For example, when the spinner tip 40 is rotated while aspiration is also applied, the clot may be reduced in as little as five seconds or less. If desired, the spinner tip 40 may be rotated to mechanically break down the residual fiber network 94, which may be aspirated into the lumen 26 by the vacuum. In addition or alternatively, the spinner tip 40 may be retracted to draw the residual fiber network 94 into the lumen 26, e.g., using one or both of the localized suction generated by the spinner tip 40 and vacuum within the lumen 26. In this alternative, the catheter 20 may be removed from the blood vessel 90 once the residual fiber network 94 is captured, e.g., withdrawn entirely from the patient's body. As described above, the methods described herein throughout may also utilize devices having one or more stops for safety.

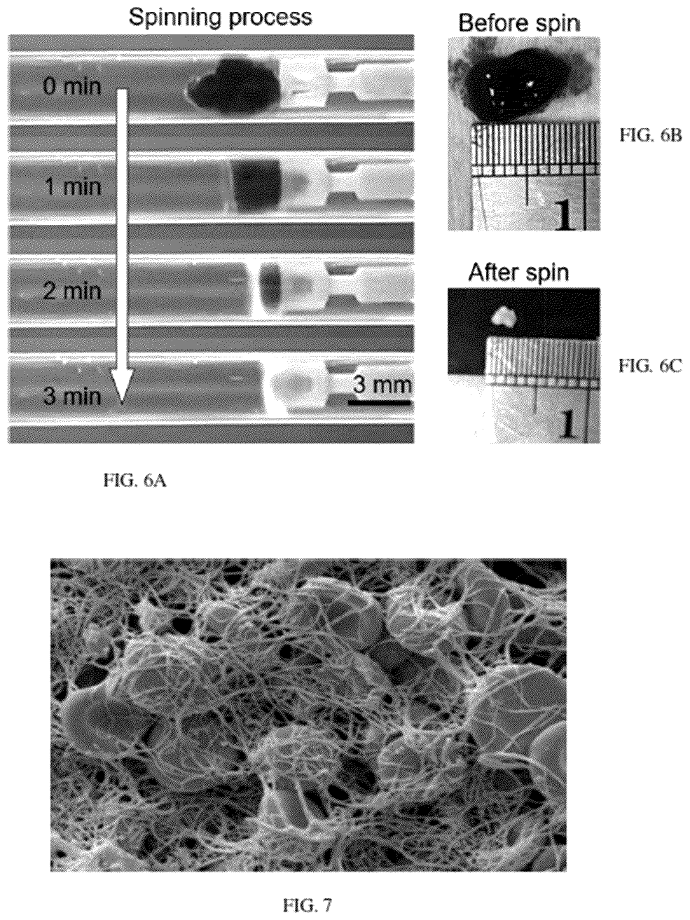

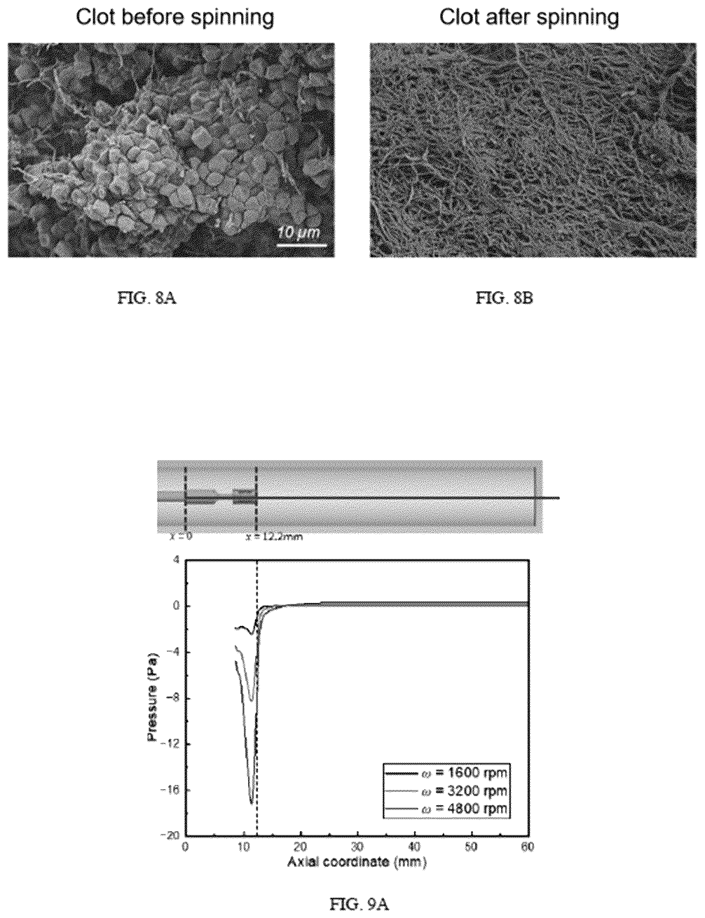

FIGS. 6A-6C show an example of the changes in a blood clot (created by pig blood in this example) during spinning the spinner tip of a thrombectomy device from zero to three minutes (0-3 min). As can be clearly seen, the size of the clot significantly reduces, and the clot color turns from red to white, as shown in FIGS. 6B and 6C. This is due to the shear force created by the spin-suction that effectively spins out all red blood cells (RBCs) from the original clot. Since a blood clot is mainly composed of RBCs trapped in fibrin fiber networks, e.g., as shown in FIG. 7, removing RBCs leaves only fibrin fiber network that is less than 10% of the initial clot volume. For example, FIGS. 8A and 8B include SEM images of a clot before and after spinning, respectively, further showing that the original clot is RBC rich and the clot after spin is highly densified fibrin fiber network.

Instead of having a sharp blade that rotates under high rpms (e.g., 150,000 to 200,000) for maceration, the spinner tip may be flexible or semi-rigid and operate under relatively low rpms (e.g., between about 4000 to 50,000) for the separation of red blood cells from fibrin network. The hole and cut features of the spinner may enhance suction to firmly compress the clot against the spinner tip distal face to ensure maximum shearing of the clot.

FIGS. 9A-9C show examples of the performance of various spinner tips under different conditions from simulations, e.g., involving positioning a spinner device within a tube, e.g., a three-millimeter tube, used to simulate operation of the spinner tip within a blood vessel. For example, FIG. 9A shows localized suction that may be generated when a spinner tip is rotated at different speeds. The localized pressure drop represents the magnitude of compression force generated, which can be tuned by varying spinning speed, thereby indicating that the clot dissolution efficiency is adjustable and may be improved with a higher spinning speed. FIG. 9B shows examples of centerline pressure profile of a spinner device with 0.87 normalized blade length (blade length, L normalized against inner radius, r) at spinning speed of 10k, 20k, 30k, and 40k rpm. FIG. 9C compares various blade lengths at 40k rpm spinning speed. The simulation was conducted for the optimization of spinner suction capability as demonstrated in FIG. 9C against normalized blade length. Optimized suction may be achieved when the normalized blade length is 0.87 and is chosen as the geometry design of the spinner tip. FIGS. 10(1) to 10(10) show additional examples of pressure distribution at the centerline of a spinner device (shown in FIG. 10) with different blade sizes during spinning.

Optionally, as shown in FIG. 4D, a drug delivery member 49 may be provided within the cavity 48 of the spinner tip 40. For example, a cylindrical body preloaded with one or more desired agents may be inserted into the spinner tip 40 immediately before a procedure or may be provided within the spinner tip 40 at the time of manufacturing. Alternatively, one or more agents may be loaded into the drug delivery member 49 immediately before the procedure and inserted into the cavity 48 to deliver the agent(s) during the procedure. For example, as described elsewhere, the agent(s) may be released as the spinner tip 40 is rotated and, optionally, the release speed of the agent(s) may be tuned to the speed of rotation of the spinner tip 40. In another option, fluid, e.g., saline and the like, may be introduced through the lumen 26 of the catheter 20 to facilitate releasing the agent(s) into the blood vessel from the spinner tip 40, if desired.

Optionally, with additional reference to FIG. 3, the controller 62 coupled to the motor 60 may be used to adjust a rotation speed of the shaft 28, e.g., to control a release rate of the one or more agents carried by a drug delivery member, e.g., one or more of clot-dissolving agents, thinning agents, anti-inflammatory agents, dyes, contrast, and/or other therapeutic and/or diagnostic agents. For example, FIG. 14 shows experimental results of controlled drug release (such as releasing the clot-dissolving medicine tPA (tissue plasminogen activator), the first treatment for acute ischemic stroke) using a spinner tip, showing the drug release rate under various spinning speeds. Under relative low spinning speeds, e.g., around ten thousand rpm, the drug release rate (expressed as a percentage of drug stored within the spinner tip that gets released per second) is lower than the release rate when the spinner tip is rotated at high spinning speeds, e.g., around forty thousand rpm. As represented by FIGS. 15A and 15B, the released dye color intensity demonstrates the different release rates, e.g., with the greater intensity shown in FIG. 15A demonstrating a faster release rate at forty thousand rpm, and the lighter intensity shown in FIG. 15B demonstrating a slower release rate. Thus, an operator may manually adjust the speed to control the release rate, and/or the controller 62 may be configured to automatically adjust the speed to provide a predetermined release rate.

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

Before the examples are described, it is to be understood that the invention is not limited to particular examples described, as such may, of course, vary. It is also to be understood that the terminology used herein is for the purpose of describing particular examples only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials are now described.

It must be noted that as used herein and in the appended claims, the singular forms “a,” “an,” and “the” include plural referents unless the context clearly dictates otherwise. Thus, for example, reference to “a compound” includes a plurality of such compounds and reference to “the polymer” includes reference to one or more polymers and equivalents thereof known to those skilled in the art, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

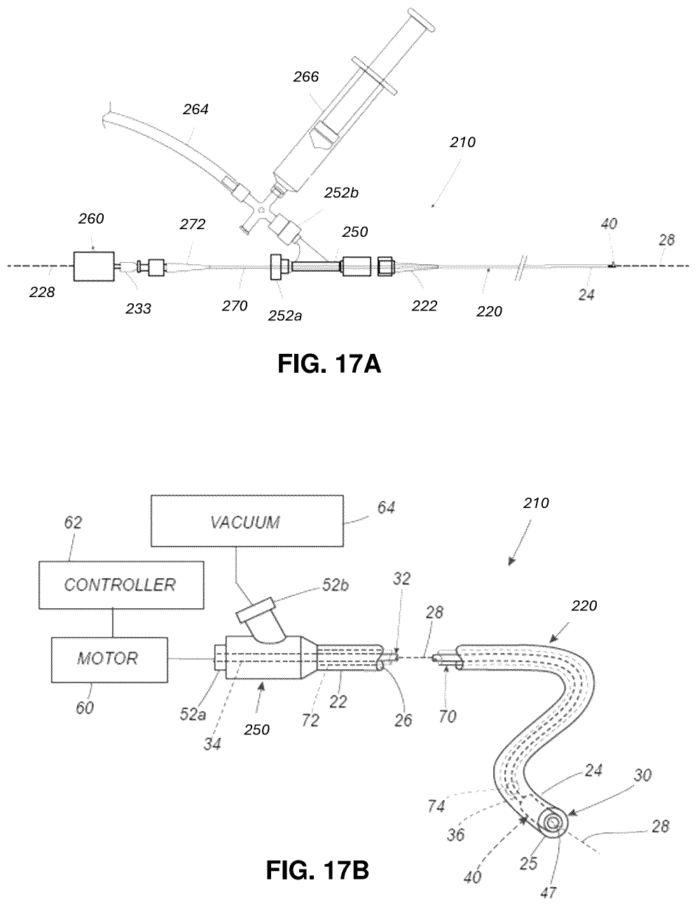

Turning to the drawings, FIGS. 17A and 17B show an example of a thrombectomy device or apparatus 10 that may be used with the systems and methods described herein throughout. In general, apparatus 10 includes an aspiration catheter, sheath, sleeve, or other outer tubular member 20, and a spinner device 30 including a spinner head or tip 40 coupled to a shaft 32 (shown in FIG. 17B) that may be introduced through, advanced along, or otherwise positioned within a lumen 26 of the catheter 20. As described further elsewhere herein, the catheter 20 and spinner device 30 may include one or more cooperating stops that limit distal movement of the spinner device 40 within the catheter 20, e.g., to prevent spinner head 40 from being exposed beyond a distal end 24 of the catheter 20 and/or to ensure the spinner head 40 is positioned to achieve maximum dissolving and/or removal of clot. In addition or alternatively, the spinner devices herein may include a two-way locking mechanism, e.g., limiting advancement of the spinner device to a distal-most position within the catheter, whereupon the spinner device may be axially fixed relative to the catheter, as described further elsewhere herein.

Optionally, the spinner device 30 may include an outer sleeve 70, which may be positioned and/or otherwise provided around the shaft 32. The sleeve 70 may help protect the inner surface of the catheter 20 when the shaft 32 rotates at high speeds, may reduce vibration resulting from rotation of the shaft 32 and/or spinner device 30, and/or may facilitate centering and/or stabilizing the spinner device 30 within the catheter 20.

Generally, as shown, the catheter 20 is an elongate tubular member including a proximal end 22 including a handle or hub 50, a distal end 24 sized for introduction into a blood vessel or other body lumen, and one or more lumens 26 extending between the proximal and distal ends 22, 24, e.g., along a longitudinal axis 28. For example, as shown, a main lumen 26 may be provided that communicates with one or more ports 52 in the handle 50 and extends to an outlet 25 in the distal end 24. Optionally, the catheter 20 may include one or more additional lumens extending at least partially between the proximal and distal ends 22, 24, e.g., a guidewire lumen for receiving a guidewire or other rail, a steering element lumen, and the like (not shown). It should also be understood that while catheter 20 is shown here as being tubular, it need not have a perfect circular cross-section. Indeed, it may be partially tubular, or have any other suitable geometry. Similarly, when reference is made to a lumen, it should be understood that a lumen may be a partial lumen, groove, or slit.

The catheter 20 may be constructed using conventional biocompatible materials and/or methods, e.g., formed from plastic, various polymers, metal, composite materials, having a substantially homogenous construction between the proximal and distal ends 22, 24. Alternatively, the construction may vary along the length of the catheter 20 to provide desired properties, e.g., to provide a proximal portion that is substantially rigid or semi-rigid, e.g., providing sufficient column strength to allow the distal end 24 of the catheter 20 to be pushed or otherwise manipulated from the proximal end 22, while a distal portion adjacent the distal end 24 may be substantially flexible to facilitate advancement through tortuous anatomy. In either construction, the catheter 20 may also be coated or layered (e.g., with a lubricious material) to aid in advancement.

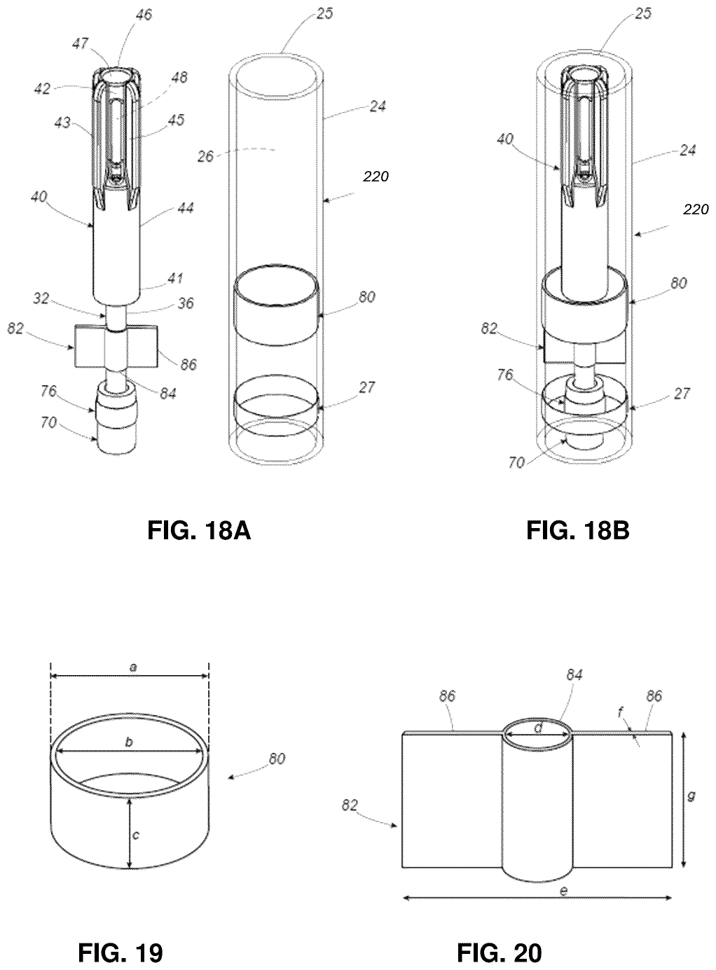

With additional reference to FIGS. 18A and 18B, the catheter 20 may include one or more stops, e.g., an annular stop 80 fixed within the lumen 26 adjacent the distal end 24, which may cooperate with one or more stops on the spinner device 30 to limit distal movement of the spinner device 30. For example, FIG. 19 shows an exemplary annular stop, i.e., a ring 80 that may be axially fixed within the lumen 26 at a predetermined distance from the outlet 25 The ring 80 may be permanently attached to the catheter 20, e.g., by one or more of gluing, hot pressing, bonding, fusing, interference fit, and the like, inside the lumen 26 near the distal end 24 with the centerline axis aligned. The ring 80 may be constructed to have an outer diameter (OD) “a” that is substantially the same as or smaller (e.g., less than 0.1 mm smaller) than the inner diameter of the catheter lumen 26. The ring 80 also has an inner diameter (ID) “b” that is sized to be larger (e.g., as larger as possible) than the outer cross-section of the spinner head 40. Consequently, when the shaft 32 and spinner head 40 are advanced distally within the lumen 26, the spinner head 40 may pass freely through the ring 80. The length “c” of the ring 80 may be relatively be short (e.g., between about half and one millimeter (0.5-1.0 mm)), e.g., to minimize the ring 80 influencing the stiffness of the catheter 20 so that the trackability and pushability of the catheter 20 is not be affected substantially.

The shaft 32 of the spinner device 30 may be an elongated flexible member including a proximal end 34 and a distal end 36 sized to be received within the lumen 26 of the catheter 20. The shaft 32 may be sufficiently long to allow introduction of the distal end 36 into a target blood vessel, e.g., through or along with the catheter 20, while the proximal end 34 remains outside the patient's body. For example, the shaft 32 may be a solid or tubular cable, e.g., including a plurality of helically wound inelastic fibers, wires, and the like, constructed to translate rotation from the proximal end 34 to the distal end 36 to rotate the spinner head 40, e.g., with sufficient torsional strength such that rotation of the proximal end 34 causes directly corresponding rotation of the distal end 36, and consequently the spinner head 40 at relatively high speeds, when coupled to a motor 60 and/or controller 62, as described further elsewhere herein as described elsewhere herein.

The outer sleeve 70 may be provided around the shaft 32, e.g., to prevent the shaft 32 from contacting the inner wall of the catheter 20 when introduced into the lumen 26 and rotated by the motor 60. The sleeve 70 may be formed from lubricious material, e.g., PTFE, and/or may include a coating on an inner surface thereof to reduce friction and/or otherwise facilitate the shaft 32 rotating in the sleeve 70. The sleeve 70 may be axially fixed relative to the shaft 32, e.g., such that the sleeve 70 extends from the proximal end 34 to the distal end 36 immediately proximal to the spinner head 40. Alternatively, the sleeve 70 may be separate from the spinner device 30, e.g., such that the sleeve 70 may be introduced into the lumen 26 of the catheter 20 before introducing the spinner head 40 and shaft 32.

Generally, as shown in FIG. 18A, the spinner head 40 may include a cylindrical or other annular body 42 including a closed proximal end 44 that may be connected to the shaft 32, and an open distal end 46 including an inlet 47 communicating with an interior cavity 48 of the body 42. Optionally, a hub 41 may be provided on the proximal end 44, which may facilitate attaching the spinner head 40 to the distal end 36 of the shaft 32. For example, the hub 41 may be used to attach the spinner head 40 substantially permanently to the shaft 32, e.g., by one or more of over-molding, fusing, sonic welding, cooperating connectors, and the like. For example, as shown, the hub 41 may include a recess that may receive the distal end 36 of the shaft 32, and the hub 41 may be bonded, melted, press-fit, and/or otherwise permanently attached over the shaft 32.

Optionally, the spinner device 30 (or any of the other examples described herein) may include one or more markers, e.g., radiopaque rings or deposited material, at desired locations, e.g., on the distal end 36 of the shaft 32 and/or on the spinner head 40, which may facilitate monitoring introduction and/or operation of the device 30 during a procedure, e.g., using fluoroscopy, Xray, ultrasound, or other external imaging. In addition or alternatively, the shaft 32 may be constructed from radiopaque materials, which may facilitate monitoring the shaft 32 during introduction and/or manipulation during a procedure.

For example, as best seen in FIG. 5A, a radiopaque marker band 76 may be provided on the outer sleeve 70, e.g., immediately adjacent a distal tip of the distal end 74. The marker band 76 may be substantially permanently attached to the distal 74, e.g., by one or more of bonding with adhesive, fusing, sonic welding, interference fit, and the like to the outer surface and/or inner surface of the sleeve 70, or may be at least partially embedded within the material of the sleeve 70.

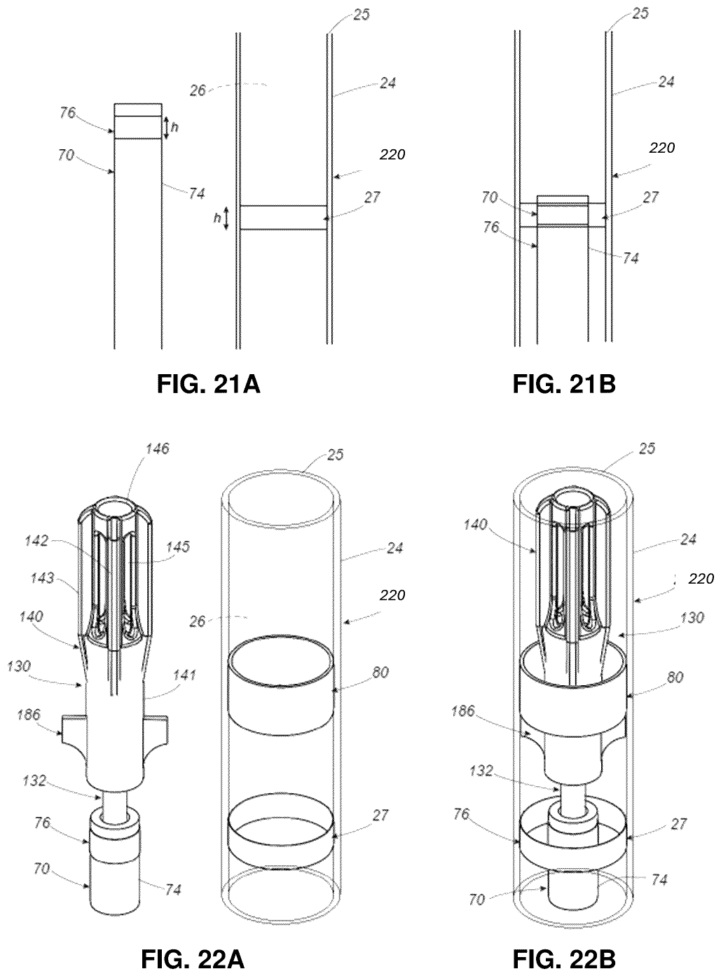

A corresponding marker may be provided on the distal end 24 of the catheter 20. For example, as shown in FIG. 21A, another radiopaque marker band 27 may be similarly permanently attached to the catheter 20, e.g., inside the catheter lumen 26, on an outer surface of the catheter 20, or within the catheter wall near the distal end 24. As described further elsewhere herein, these marker bands 76, 27 may facilitate monitoring the device 10 under x-ray and/or other external imaging, e.g., to visually examine whether the spinner head 40 has been advanced sufficiently within the lumen 26, i.e., positioned immediately adjacent the outlet 25. For example, the axial location of the catheter marker band 27 may be set such that, when a distal end 46 of the spinner head 40 is positioned immediately adjacent the outlet 25 within the lumen 26, the sleeve marker band 76 may be axially aligned with the catheter marker band 27, e.g., as shown in FIGS. 18B and 21B. Optionally these two marker bands 76, 27 may have the same axial length or height “h,” e.g., as shown in FIGS. 21A and 21B, e.g., between about one and two millimeters (1.0-2.0 mm). The marker bands 76, 27 may have a thickness that is extremely thin, e.g., less than 0.1 millimeters, such that the bands 76, 27 do not interference with manipulation of the spinner device 30 within the lumen 26. Although FIGS. 21A and 21B include the spinner device 30 shown in FIGS. 18A and 18B, similar markers may be provided in the spinner devices and/or systems described elsewhere herein.

With particular reference to FIGS. 18A and 18B, an exemplary spinner head 40 is shown that may be used with the devices, systems and methods provided herein, e.g., on the distal end 36 of the shaft 32. Additional examples of spinner heads that may be provided on the devices and systems herein may be found in the applications incorporated by reference elsewhere herein. For example, as shown, the spinner head 40 may include a cylindrical spinner body 42 that includes a substantially uniform annular wall extending between the proximal end 44 and the distal end 46, which includes inlet 47 communicating with the interior cavity 48. The distal end 46 of the spinner head 40 may be rounded as shown, e.g., to provide a substantially atraumatic distal face for the spinner head 40. Alternatively, the distal end 46 may include a substantially flat distal face (not shown), which may facilitate placement against clot positioned immediately adjacent the outlet 25 of the catheter 20 without macerating the clot when the spinner head 40 is rotated.

Optionally, as shown, the spinner head 40 may include one or more blades, struts, or other exterior features on the spinner body 42 to enhance localized suction and/or shear force on the clot, e.g., a plurality of elongate blades 43 extending at least partially between the proximal and distal ends 44, 46 of the spinner body 42. As shown, four blades 43 are shown that are spaced evenly around the spinner body 42, e.g., offset about ninety degrees from one another around the circumference of the spinner head 40. Also as shown, the blades 43 may extend the length of the spinner body 42 or may only extend partially between the proximal and distal ends 44, 46. It should be understood that while equal spacing is shown here, unequal spacing is also contemplated. In addition, while four blades are 43 shown, any number of blades may be used, e.g., two, three, four, five, six, seven, etc. Similarly, it should be understood that the blades need not be of equal dimension (e.g., length, width, height). In some variations, the blades may be of unequal dimensions.

In addition or alternatively, as shown, the spinner head 40 may include one or more slits or other openings in the wall of the spinner body 42, e.g., extending radially outwardly from the cavity 48 through the wall to the outer surface of the spinner body 42. For example, as shown, an elongate slit 45 is provided between each adjacent blade 43, the slit 45 extending partially between the proximal and distal ends 44, 46 of the spinner body 42. The slits 45 may enhance the localized suction within the cavity 48 and inlet 47, e.g., as described elsewhere herein and in the applications incorporated by reference herein. Alternatively, slits may be provided only between some of the blades and/or a plurality of slits may be provided in a cylindrical body without blades (not shown), as desired. Although four slits and blades are shown, it will be appreciated that any desired number of slits and/or blades may be provided on the spinner head 40 spaced around the annular body 42, e.g., two, three, four, or more. Similarly, it should be appreciated that the slits need not be of equal size. Adding blades and adding both blades and slits to the spinner head may substantially increase the localized suction that is generated by the spinner head 40.

In some examples, the spinner body 42 may have a length between about one to five millimeters (1.0-5.0 mm), e.g., about 4.2 mm, and a wall thickness between about 0.05-0.15 mm, e.g., about 0.09 mm or 0.15 mm; the inlet 47 may have a diameter between about 0.5-1.2 mm, e.g., about 0.72 mm or 1.2 mm. The blades 43 may have heights between about 0.1-0.7 mm, e.g., about 0.31 mm or 0.51 mm, and widths between about 0.1-0.5 mm, e.g., about 0.24 mm or 0.40 mm. The slits 45 may have lengths between about 0.5-2.5 mm, e.g., about 2.1 mm, and widths between about 0.2-0.8 mm around the radius, e.g., about 0.45 mm or 0.75 mm.

In another alternative, the spinner head may include a plurality of helical blades (not shown), e.g., extending at least partially around the circumference of the annular body and/or along the length of the annular body. Optionally, in this alternative, one or more helical slits or other openings may be provided between one or more of the helical blades. It will be appreciated that one or more additional features may be provided on the outer surface of the annular body, in addition to or instead of the blades and/or slits, that may enhance the localized suction generated by the spinner head.

With continued reference to FIGS. 18A and 18B, the spinner head 40 (or any other examples described herein) may be integrally formed as a single piece, e.g., from plastic, metal, composite material, and the like, e.g., manufactured using one or more of 3D-printing, by molding, micro-injection molding, casting, machining, and the like. Alternatively, the spinner body 42 and/or hub 41 may be formed from one or more substantially continuous processes, such as extrusion and the like, e.g., to allow manufacturing a single assembly that may be separated into individual tips. Optionally, the spinner body 42 may be formed without the slits and/or blades, which may be added or formed during subsequent processing of the annular body, if desired.

The spinner head 40 may be substantially rigid or, alternatively, the material of the spinner head 40 may be flexible or semi-rigid, e.g., formed from relatively soft material, such as elastomeric material, e.g., silicone, or soft plastics, such that the distal end 46 of the spinner body 42 provides a substantially atraumatic tip that may minimize risk of damaging tissue contacted by the tip. For example, the spinner head 40 may be formed from relatively soft material, such as an elastomeric material with stiffness less than 40 MPa. Thus, the spinner head 40 may be able to recover from deformation and retain its shape, e.g., by bending less than about one hundred eighty degrees (180°) degrees and/or twisting less than about five hundred forty degrees (540°). Similarly, the blades 43 may be formed from flexible and/or soft material and/or may include rounded or other atraumatic outer edges, e.g., to prevent damaging the vessel wall and/or causing the clot to be macerated when the spinner head 40 is rotated.

The spinner head 40 may have a diameter or other outer cross-section sized to be received within the lumen 26 of the catheter 20 while allowing the spinner head 40 to rotate freely, e.g., providing clearance around the spinner head 40 within the lumen 26. For example, the spinner head 40 may have an outer diameter or other cross-section between about 1.2-2.5 millimeters, e.g., having an outer diameter of about two millimeters (2 mm) or less.

With additional reference to FIGS. 17A and 17B, the spinner head 40 is connected to the shaft 32, which is coupled, in turn, to a motor 60 that provides the torque to rotate the spinner head 40. For example, the proximal end 34 of the shaft 32 may include a connector (not shown) to couple the shaft 32 to the motor 60, e.g., via an external drive shaft, cable, and the like (also not shown). The motor 60 may be configured to rotate the spinner head 40 at desired speeds, e.g., at least 1000 rpm, or at least 10,000 rpm, e.g., between about 1,000 and 200,000 rpm, between about 4,000 and 50,000 rpm, between about 10,000 and 40,000 rpm, between about 20,000 and 40,000 rpm, or between about 30,000 and 40,000 rpm, e.g., around 10,000 rpm or around 40,000 rpm, which may generate a desired localized suction pressure at the inlet 47 of the spinner head 40. For example, with the spinner head rotating at 40,000 rpm, localized suction of about seven thousand Pascals (7 kPa) may be generated, while at 10,000 rpm, five hundred Pascals (0.5 kPa) may be generated.

In one example, the motor 60 may be configured to rotate the spinner head 40 at a single set speed. Alternatively, the speed of the motor 60 may be variable, e.g., manually using an actuator of the controller 62 coupled to the motor 60, which may be adjusted by the user to modify the rotation speed of the spinner head 40. Alternatively, the controller 62 may be configured to initially operate the motor 60 at a relatively lower speed and then the speed may be automatically increased to rotate the spinner head 40 at a desired active speed. For example, the initial speed may be used to mechanically engage the clot, and then the speed may be increased, e.g., to rapidly remove red blood cells from the clot and reduce clot size, as described elsewhere herein.

Different from existing aspiration thrombectomy devices, which require continuous extraction of blood from the vessel being treated, the spinner devices herein may generate a highly localized suction force without removing any fluid from the vessel. Optionally, the devices may be used in conjunction with aspiration, e.g., by connecting a source of vacuum 64, e.g., a vacuum line as shown in FIG. 17A, to the catheter 20 or introducing a separate suction device (not shown), as described further elsewhere herein.

A controller 62 may be coupled to the motor 60 to control operation of the device 10, e.g., to allow a user to turn the motor 60 off and on to rotate the spinner head 40 and generate the localized suction with a blood vessel. Optionally, the controller 62 may include one or more actuators, e.g., switches and the like (not shown), to activate/deactivate the motor and/or to adjust the speed, if desired. In addition or alternatively, the controller 62 may include an actuator (also not shown) to advance and/or retract the shaft 32 axially, e.g., relative to the catheter 20, e.g., using an advancer device (not shown), such as that shown in the applications incorporated by reference herein. In another option, the controller 62 may include a robotic control system to control axial movement of the shaft 32 remotely, if desired.

In one example, the spinner device 30 and catheter 20 are assembled together, e.g., such that the catheter 20 and spinner device 30 are introduced together into a patient's body, e.g., with the spinner head 40 initially positioned proximal to the outlet 25 of the catheter 20. In this way, the devices described herein are part of a pre-assembled system or kit. Alternatively, the spinner device 30 may be separate from the catheter 20, i.e., including the shaft 32 and spinner head 40, e.g., such that the catheter 20 may be introduced initially into a patient's body, and, once the distal end 24 and outlet 25 are positioned adjacent a target clot, the spinner device 30 may be introduced into the catheter 20 and advanced to position the spinner head 40 adjacent the outlet 25. In this way, the devices described herein may be assembled just prior to, or during, use. In this example, the handle 50 of the catheter 20 may include a port 52a that allows the spinner device 30 to be inserted into and removed from the lumen 26 of the catheter 20, which may include one or more hemostatic seals, e.g., to prevent fluid from leaking from the port 52a while allowing the shaft 32 of the spinner device 30 to be advanced through the port 52a into the lumen 26. Alternatively, the spinner device 30 may be permanently integrated with the catheter 20, e.g., such that the spinner device 30 cannot be removed but may be advanced and/or retracted axially within the lumen 26, e.g., using the advancer device

Turning to FIGS. 18A and 18B, a first example of a set of stops on the spinner device 30 are shown that may interact with the annular stop 80 to prevent the spinner device 30 from being advanced to expose the spinner head 40 from the outlet 25 of the catheter 20. In this example, the spinner device 30 includes a wing-stopper 82 including a hollow cylinder or other base 84 from which a plurality of wings, arms, beams, or other elements 86 extend radially outwardly. As shown, the wing-stopper 82 includes a pair of wings 86 extending from the base 84 opposite one another, e.g., disposed about one hundred eighty degrees (180°) from one another around the circumference of the base 84 such that the wings 86 lie within a common plane. It will be appreciated that the wing-stopper 82 may include additional wings, e.g., a total of three, four, or more, which may be disposed uniformly or non-uniformly around the circumference, as desired.

The base 84 may be attached to or otherwise axially and/or circumferentially fixed to the rotation shaft 32, e.g., attached to the distal end 36 of the shaft 32 proximal to the spinner head 40. The base 84 may be substantially permanently attached to the distal end 36 of the shaft 32 by one or more of bonding with adhesive, sonic welding, fusing, interference fit, and the like such that the base 84, and consequently the wing 86, rotate when the shaft 32 rotates.

In the example shown in FIG. 20, the cylinder or base 84 of the wing-stopper 82 has an inner diameter “d” that is substantially the same as or larger (e.g., less than 0.1 millimeter larger) than the outer diameter (“OD”) of the rotation shaft 32, allowing the base 84 to be fixed on the rotation shaft 32. The radial length of the wings 86 “e” may be slightly smaller (e.g., less than 0.1 millimeter smaller) than the inner diameter of the lumen 26 of the catheter 20, e.g., to ensure a smooth pass-through catheter lumen, and larger than the inner diameter “b” of the internal ring 80. Consequently, during the spinner insertion process, because the spinner head 40 is smaller than the ring 80, the spinner head 40 may pass freely through the ring 80 until the wing-stopper 82 reaches a proximal end of the ring 80 in the catheter 20. Thus, further distal movement of the spinner head 40 is constrained, e.g., to prevent exposure of the spinner head 40 beyond the distal end 24 of the catheter. The thickness “f” of each wing 86 may be set to minimize blockage of aspiration of clot or other debris into the lumen 26, e.g., to maximize an open cross-section of the lumen 26 through the wing-stopper 82. Optionally, the wings 86 may have tapered, rounded, or otherwise shaped distal edges to facilitate debris passing through the lumen 26 past the wings 86. The axial length “g” of the wings 86 (and/or base 84) of the wing-stopper 82 may be minimized, e.g., (between about half and one millimeter (0.5-1.0 mm) to minimize impact on the flexibility of the rotation shaft 32 and/or deliverability of the spinner device 30.

As shown in FIGS. 18A and 18B, the axial position of the wing-stopper 82 and internal ring 80 may be predetermined relative to one another to ensure that the distal end 46 of the spinner head 40 is aligned with the distal end 24 of the catheter 20 when the wings 86 abut the proximal end of the internal ring 80. For example, the distance from the distal edge of the wings 86 of the wing-stopper 82 to the distal end 46 of the spinner head 40 may be set to be substantially the same as the distance from the proximal edge of the internal ring 80 to the outlet 25 at the distal end 24 of the catheter 20.

Optionally, as shown in FIGS. 18A and 18B, the relative distances may be set such that a portion of the rotation shaft 32 is purposely exposed between the proximal hub 41 of the spinner head 40 and the distal edge of the wings 86 of the wing-stopper 82, which may ensure good passage for dissolved and/or aspirated clot past the spinner device 30 into the lumen 26 of the catheter 20. In one example, the length of this exposed region of the rotation shaft 32 between the wing-stopper 82 and the spinner head 40 may be larger (e.g., less than 0.5 millimeter larger) than the axial length of the ring 80.

Optionally, the catheter 20 and/or shaft 32 may include one or more additional stops or other safety features (not shown) to limit axial movement of the shaft 32 and thus help prevent accidental shearing of non-clot tissue during use. For example, a stop may be provided within the handle 50 and/or on the proximal end 34 of the shaft 32 that prevents the spinner device 30 from being advanced to expose the spinner head 40 from the outlet 25 of the catheter 20. In addition or alternatively, one or more stops or locks may be provided on the handle 50 and/or shaft 32 that axially lock the shaft 32, e.g., once the spinner head 40 is advanced to its distal-most position. In addition, the one or more stops or locks may prevent the spinner head 40 from retracting away from the outlet 25 of the catheter 20, e.g., due to the localized suction forces and/or aspiration suction within the lumen 26 of the catheter 20. Thus, the distal tip 46 of the spinner head 40 may be maintained immediately adjacent the outlet 25 within the lumen 26 to maximize dissolving efficiency as the spinner head 40 rotates.

Alternatively, the stop(s) may allow the spinner head 40 to be exposed partially or entirely from the outlet 25, if desired. Optionally, another stop may be provided that allows the spinner head 40 to be retracted a desired distance proximally from the outlet 25, e.g., to allow residual fiber from a reduced or dissolved clot to be aspirated or otherwise directed into the outlet 25. In another alternative, the spinner device 30 may be permanently axially fixed relative to the catheter 20, e.g., such that the spinner head 40 is located within the lumen 26 with the distal end 46 of the spinner head 40 located immediately adjacent the outlet 25.

If the spinner device 30 is permanently integrated with the catheter 20, the proximal end 34 of the shaft 32 may extend from the port 52a on the handle 50 (whether the shaft 32 is movable axially or axially fixed), e.g., as shown in FIGS. 17A and 17B. Optionally, if the shaft 32 is hollow and/or includes a lumen, a port (not shown) may be provided on the shaft 32, e.g., on a hub 33 on the proximal end 34, which may communicate with the lumen, which may be connected to a source of vacuum and/or fluid, e.g., to aspirate and/or flush the lumen of the shaft 32.

The proximal end 34 of the shaft 32 may include a connector configured to couple the shaft 32 to a driveshaft of the motor 60, e.g., to allow the thrombectomy device 10 to be connected and disconnected from the motor 60. In this example, the thrombectomy device 10 may be a single-use, integral device that may be provided to a user for use during a procedure, after which the device 10 may be discarded. Alternatively, if the spinner device 30 is provided separately from the catheter 20, both may be single-use and/or disposable or one or both may be reusable, e.g., after cleaning and/or sterilization. In a further alternative, the spinner device 30 may be provided and/or introduced into a patient's body without the catheter 20, if desired.

Turning to FIGS. 22A and 22B, another example of a spinner head 140 is shown that may be included in the devices and systems described herein. Similar to the spinner head 40, the spinner head 140 includes an annular spinner body 142 that may include a plurality of blades 143 and/or slots 145 that may be configured to enhance localized suction adjacent a distal end 146 of the spinner head 140. Unlike the previous spinner head 40, the spinner head 140 includes a plurality of wings 186, e.g., a pair of opposing wings 186 extending from opposite sides of a hub 141 on the proximal end of the spinner head 140. The wings 186 may be integrally formed with the other features of the spinner head 140 or may be formed separately and permanently attached to the spinner head 140.

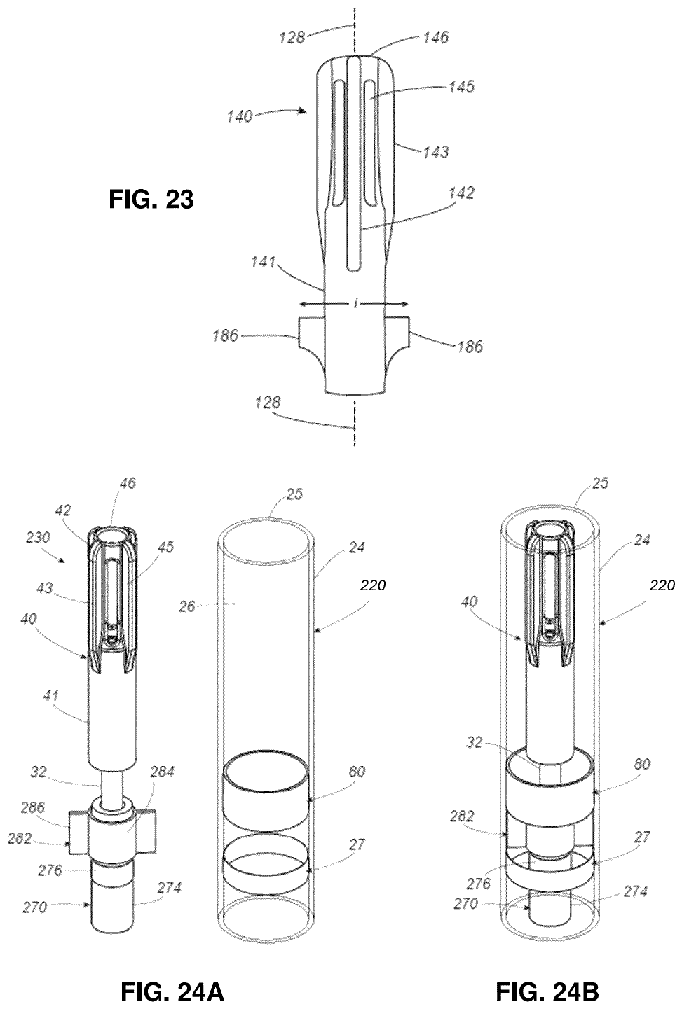

For example, as shown in FIG. 23, the spinner head 140 may be integrally formed, e.g., by molding, casting, machining, and the like, to provide all of the features of the spinner head 140. In this example, the spinner head 140 includes a pair of wings 186 on the proximal hub 141 (or alternatively, additional wings, not shown) that extend radially outwardly from the proximal hub 141, e.g., lying within a common plane. The lengths of the wings 186 may define a total length or other overall cross-section “i” substantially perpendicular to a longitudinal axis 128 of the spinner head 140 that is smaller (e.g., less than about 0.1 millimeter smaller) than or equal to the inner diameter of the lumen 26 of the catheter 20 and larger than the inner diameter of the ring 80. This configuration allows the spinner head 140, which has an outer diameter or other cross-section smaller than the inner diameter of the ring 80 to freely pass through the ring 80, e.g., as shown in FIG. 22B, while having the wings 186 blocked by the ring 80 from moving further distally once the wings 186 abut the ring 80.

Optionally, a short portion of the rotation shaft 132, e.g., less than about 0.5 millimeter, may be exposed between the distal end 74 of the outer sleeve 70 and the proximal end 141 of the spinner head 140, e.g., to prevent or minimize friction between the rotating spinner head 141 and distal end 74 of the sleeve 70. When the spinner device 130 is advanced, the distal end surfaces of the wings 186 may abut or otherwise contact with a proximal edge of the ring 80, thereby locking the spinner head 140 inside the catheter 20 and/or aligning the distal end 146 of the spinner head 140 with the outlet 25 at the distal end 24 of the catheter 20. Compared to the spinner device 30 shown in FIGS. 18A and 18B, the spinner head 130 may have fewer parts, e.g. eliminating the separate wing-stopper 80, which may potentially case the manufacturing process.

Turning to FIGS. 24A and 24B, another example of a spinner device 230 is shown that may be included with a catheter 20 to provide a thrombectomy device and/or system. Similar to the device 10, the spinner device 230 includes a rotation shaft 32 carrying a spinner head 40. In addition, the spinner device 230 includes an outer sleeve 270 that includes a wing-stopper device 282 on its distal end 274, e.g., adjacent a radiopaque marker band 276 on the sleeve 270. However, instead of providing a wing-stopper fixed on the rotation shaft, the wing-stopper 282 includes a ring or base 284 that is fixed to the outer sleeve 270. For example, the base 284 may be substantially permanently attached to the distal end 274 of the outer sleeve 270, e.g., by one or more of gluing, fusing, sonic welding, interference fit, and the like, at a desired location offset from the distal tip of the sleeve 270, e.g., about 0.1-0.2 millimeter away from the distal tip of the sleeve 270. Thus, in this example, the wing-stopper 282 is a non-rotating component, i.e., that remains fixed during rotation of the rotation shaft 32 and the spinner head 40.

The radial length or other overall cross-section of the wings 286 of the base 284 of the wing-stopper 282 may be smaller than (e.g., less than 0.1 millimeter smaller than) or equal to the inner diameter of the lumen 26 of the catheter 20 and larger than the inner diameter of the ring 80 on the catheter 20. The inner diameter of the base 284 of the wing-stopper 282 may be substantially the same as or larger (e.g., less than 0.1 millimeter larger) than the outer diameter of the sleeve 270, e.g., to allow the base 284 of the wing-stopper 282 to fit over and/or be axially fixed relative to the sleeve 270. When the spinner device 230 is advanced within the lumen 26 of the catheter 20 lumen towards the catheter distal end 24, the wings 286 may contact the ring 80 to provide a locking mechanism that prevents further distal movement of the spinner head 40.

For example, the distance from the proximal edge of the ring 80 to the distal tip at the catheter distal end 24 may be set to be substantially the same as the distance from the distal edges of the wings 286 of the wing-stopper 282 to the distal end 46 of the spinner head 40. In this way, the spinner head 40 may have its distal end 46 aligned with the outlet 25 at the distal end 24 of the catheter 20, while preventing the distal end 46 from being exposed outside the catheter 20.

Optionally, a portion of the rotation shaft 32 may be exposed between the distal end 274 of the sleeve 270 and the proximal end 41 of the spinner head 40, e.g., to minimize blockage of the lumen 26 during aspiration, e.g., having an exposed length that is substantially the same as or larger (e.g., less than about 0.5 millimeter larger) than the axial length of the ring 80. Compared to the examples shown in FIGS. 18A-18B and 22A-22B, the locking mechanism does not require a wing-stopper to rotate during operation, which may reduce the risk of component and/or system failure.

In this example, the axial location of the sleeve marker band 276 may be set differently than the previous examples. For example, the mark band 276 may be located further axially back (towards the proximal end of the sleeve 270), leaving space for installation of the wing-stopper 282. The distance from the distal tip of the sleeve 270 to the sleeve marker band 276 may be set to be larger than (e.g., less than 0.1 millimeter larger than) than the axial length of the wing-stopper 282.

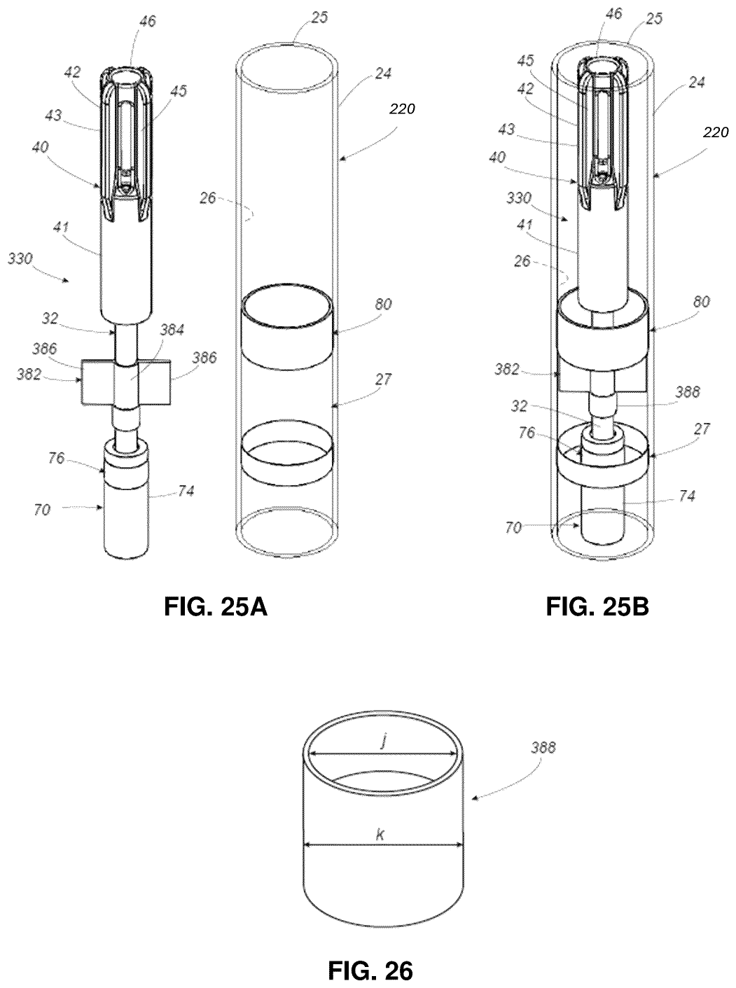

Turning to FIGS. 25A and 25B, another example of a spinner device 330 is shown that may be provided within or introduced into a catheter 20 to perform a thrombectomy procedure. Similar to the previous devices, the catheter 20 includes a proximal end (not shown), a distal end 24, a lumen 26 extending therebetween, and an annular ring 80 fixed within the lumen 26 proximal to the distal end 24. The spinner device 330 includes a rotation shaft 32 carrying a spinner head 40 and an outer sleeve 70, e.g., similar to the spinner device 30.

Unlike the spinner device 30, the spinner device 330 includes a wing-stopper 382 carried on the shaft 32 that is not coupled to the shaft 32. For example, as shown, the wing-stopper 382 may include an annular ring or other base 384 that is free-floating on the shaft 32. A pair of wings 386 (or any desired number) may be provided on the base 384 that extend radially outwardly to define an outer diameter or cross-section that is larger than the inner diameter of the ring 80, e.g., similar to other devices herein.

In addition, a blocker 388 may be provided on the rotation shaft 32, e.g., permanently attached to or otherwise axially fixed on the distal end 36 of the shaft 32, e.g., distal or otherwise adjacent to the distal end 74 of the outer sleeve 70, e.g., spaced apart a desired distance from the distal end 74. For example, as shown in FIG. 26, the blocker 388 may include a hollow cylinder with an inner diameter “j” that is substantially the same as or larger than (e.g., less than 0.1 millimeter larger than) than the outer diameter of the rotation shaft 32, e.g., such that the blocker 388 may be permanently attached to the shaft 32, e.g., by one or more of bonding with adhesive, fusing, sonic welding crimping, interference fit, and the like. The blocker 388 also has an outer diameter “k” that is larger than (e.g., about 0.1-0.2 millimeter larger than) the inner diameter of the base 384 of the wing-stopped 382 such that the blocker 388 may prevent proximal movement of the wing-stopper 382 towards the distal end 74 of the outer sleeve 70. Thus, the blocker 388 may be axially and/or rotationally fixed on the rotation shaft 32 whereas the wing-stopper 382 is not fixed on the rotation shaft 32, meaning that the wing-stopper 382 may not rotate with the rotation shaft 32 during operation.

Optionally, a portion of rotation shaft 32 may be exposed, e.g., between the base 384 of the wing-stopper 382 and the proximal end 41 of the spinner head 40, to ensure good passage of dissolved and aspirated clot into the lumen 26 of the catheter around the spinner device 330.

The length of the exposed region may be set to be larger than (e.g., less than 0.5 millimeter larger than) the axial length of the ring 80. Similar to other devices herein, the axial distance from the distal edges of the wings 386 of the wing-stopper 382 to the distal end 46 of the spinner head 40 may be set to be substantially the same as the distance from the proximal edge of the ring 80 to the outlet 25 in the distal end 24 of the catheter 20. For example, FIG. 25B shows the exemplary configuration where when the wing-stopper 382 intersects with the ring 80, thereby aligning the spinner head 40 with the outlet 25 at the distal end 24 of the catheter 20 and preventing further distal movement. Compared to previous examples, the configuration of the spinner device 330 may prevent the wing-stopper 382 from rotating during operation. For example, while the shaft 32 rotates within the base 384 of the wing-stopper 382, the wing-stopper 382 may float freely without substantial rotation and the blocker 388 may prevent proximal movement of the wing-stopper 382.

The arrangement of the free-floating wing-stopper 382 over the rotation shaft 32 requires a physical interaction between rotating and non-rotating parts to achieve position constraint functionality. The friction induced during this interaction may affect spinner spinning performance, which may lead to a decrease in milli-spinner clot removal efficiency. To overcome such inefficiencies and/or reduce frictional forces, the geometry design of the wing-stopper and blocker may be modified to minimize friction between these components.

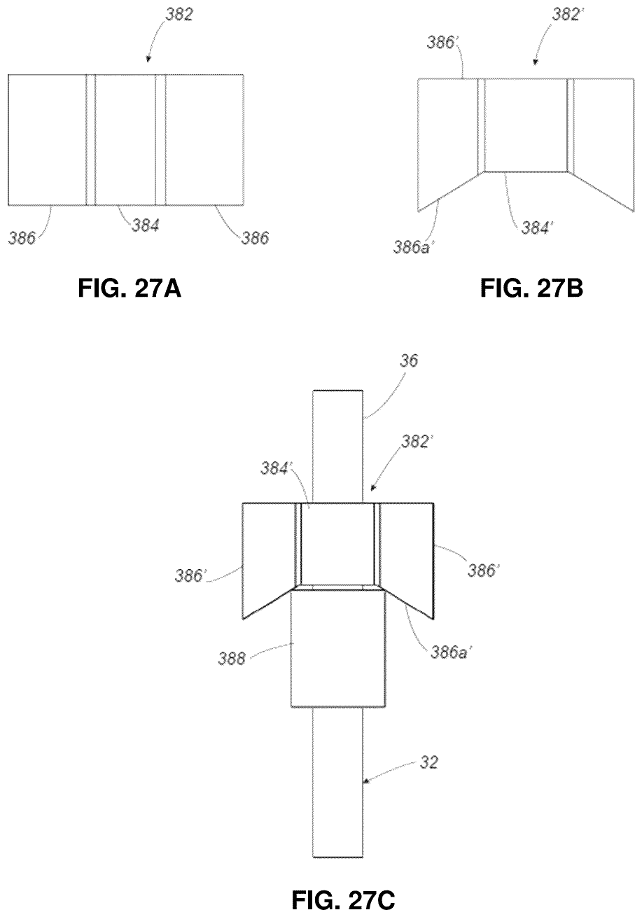

One way to reduce friction between rotating and non-rotating components is to decrease their interface area. For example, as shown in FIGS. 27B and 27C, a modified wing-stopper 382′ may be provided, which may be include an annular ring or base 384′ free-floating on the rotation shaft 32. Unlike the wing-stopper 382 shown in FIG. 27A, the wings 386′ of the wing-stopper 382′ may be modified, e.g. by cutting or otherwise shaping proximal edges 386a of the wings to minimize contact with the blocker 388 (which rotates with the rotation shaft 32). To achieve this, the outer diameter of the blocker 388 may be sized to be larger than the outer diameter of the cylinder base 384′ of the modified wing-stopper 382′ and the proximal edges 386a′ of the wings 386′ may be tapered such that the edges 386a′ only contact an outer distal edge of the blocker 388, which may reduce friction between the wings 386′ and the blocker 388. Optionally, a similar design may be applied to the wing-stopper 82 and the sleeve 70 of the spinner device 30 shown in FIGS. 18A and 18B.

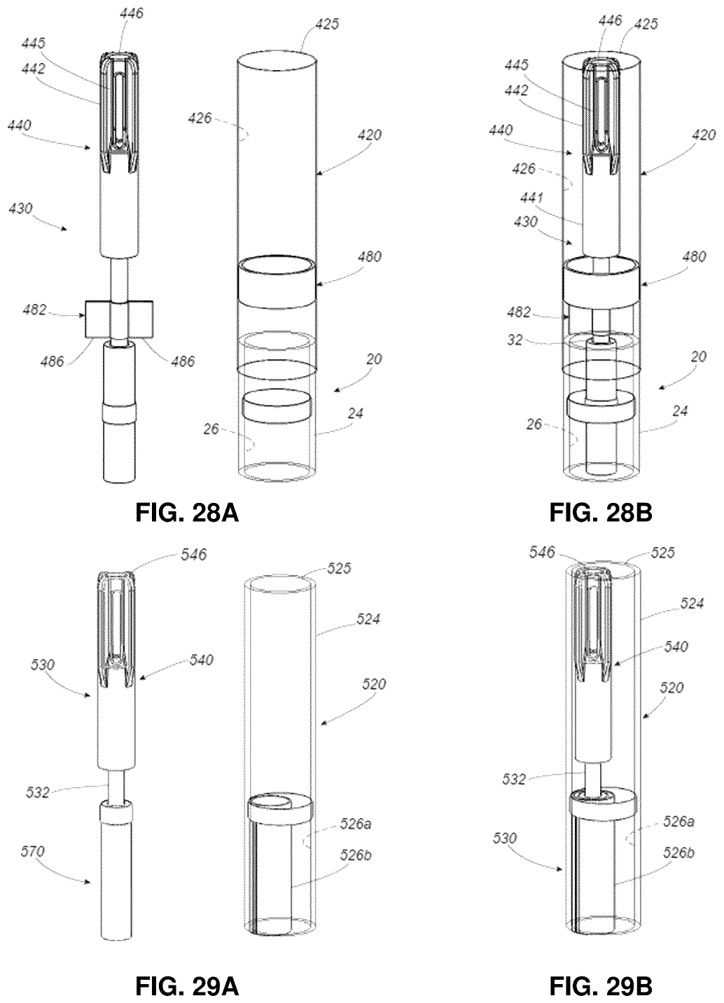

Optionally, rather than attach an internal ring within the catheter adjacent the outlet, as shown in FIGS. 28A and 28B, a separate sleeve or tubular section 420 may be attached to the distal end 24 of the catheter 20. The tubular section 420 may include an annular ridge, bump, or one or more other features 480 that extends inwardly into the passage 426 through the sleeve 420 that is sized to interact with a wing-stopper 482 on the spinner device 430. For example, the feature(s) 480 may be sized such that the spinner head 440 may pass freely through the tubular section 420, while the wings 486 of the wing-stopper 482 may abut the feature(s) 480, thereby limiting further distal movement of the spinner head 440, e.g., to prevent the spinner head 440 from being exposed beyond the tubular section 420, similar to the previous devices.

Alternatively, in any of the examples described above, it will be appreciated that the components may be reversed. For example, a ring (not shown) may be mounted on the spinner device, e.g., on one of the spinner head, rotation shaft, and/or outer sleeve, instead of the wing-stopper described above, e.g., similar to the device shown in FIGS. 19A and 19B. The ring may be concentrically mounted around the spinner device, e.g., by one or more radial struts or other supports that axially fix the ring. The ring may be sized to slide along an inner surface of the catheter, e.g., to allow the spinner head to be advanced within the catheter, e.g., until the distal tip of the spinner head is disposed adjacent the outlet of the catheter. In this alternative, the catheter may include one or more internal features, e.g., wings, tabs, bumps, and the like, that extend inwardly into the catheter lumen. The features may be sized to allow the spinner head to pass distally beyond the features but interact with the ring, e.g., to prevent further distal movement of the spinner head when the ring contacts the features.

In other alternatives, the devices and systems may include other one-way or two-way stopping or locking mechanisms that limit axial movement of the spinner device relative to a delivery catheter or other tubular member. For example, turning to FIGS. 29A and 29B, the device may include an aspiration catheter 520 including a primary lumen 526a, e.g., for providing suction to aspirate material into the catheter 520, and a secondary lumen 526b within which the spinner device 530 may be slidably received, e.g., such that the spinner head 540 may be positioned adjacent the outlet 525 of the catheter 520. The spinner device 530 and catheter 520 may include cooperating stops (not shown), e.g., at their proximal ends that may limit distal advancement of the spinner head 540, e.g., to prevent the spinner head 540 from being exposed beyond the outlet 525.

The catheter 520 and the rotation shaft 532 of the spinner device 530 may have corresponding lengths such that, when the stops interact to limit further advancement, the distal tip 546 of the spinner head 540 is positioned immediately adjacent the outlet 525 within the primary lumen 526a. With the spinner device 530 received within the secondary lumen 526b, the shaft 532 may follow the path of the secondary lumen 526b even when the catheter 520 is introduced through tortuous anatomy to maintain the axial location of the spinner head 540 relative to the outlet 525. For example, the diameter of the secondary lumen 526b may constrain the spinner device 530 from lateral motion within the catheter 520 and, the relative lengths of the spinner device 530 and catheter 520 may be set to precisely position the spinner head 540 immediately adjacent the outlet 525 when everything is aligned in a straight configuration. Consequently, no matter how the catheter 520 is deflected, e.g., when directed through tortuous anatomy, the shaft 532 and sleeve 570 will always follow the curvature of the secondary lumen 526b such that the spinner head 540 does not move axially relative to the outlet 525.

Turning to FIGS. 30A and 30B, another exemplary spinner device 630 and aspiration catheter 620 are shown, which may be generally similar to other devices herein. In this example, however, the outer sleeve 670 of the spinner device 630 includes a plurality of balloons 678 configured to expand and contact the inner wall of the lumen 626 of the catheter 620, e.g., to center the spinner head 640 within the lumen 626. For example, a plurality of radial balloons 678 may be attached to the outer sleeve 670 that may be initially collapsed and inflated when desired, e.g., such that the balloons 678 extend outwardly to slidably contact the catheter 620, as shown in FIG. 14C, thereby centering the outer sleeve 670, and consequently, the spinner head 640. Although only a single set of balloons 678 is shown, optionally, multiple sets of balloons may be provided on the outer sleeve 670, e.g., spaced apart axially from one another (not shown). The outer sleeve 670 may include one or more inflation lumens (not shown) that communicate with interiors of the balloons 678 and extend to a proximal end of the sleeve 670, e.g., communicating with a port on the handle or hub (also not shown) of the spinner device 640. Thus, a source of inflation media, e.g., a syringe and the like) may be used to deliver inflation fluid into the balloons via the lumen(s) and evacuate the fluid to inflate and deflate the balloons, as desired.

Given the relative lengths of the shaft 632 and the catheter 620, the balloons 678 may ensure that the spinner head 640 is positioned immediately adjacent the outlet 625 when stops limit further distal advancement of the spinner device. For example, the balloons 678 may align the spinner device along the centerline of the catheter 620 to maintain the spinner head 640 adjacent the outlet 625 even if the catheter 620 is directed through tortuous anatomy (e.g., as opposed to a loose sleeve, which may move laterally within the catheter 620 when the catheter is directed through multiple bends, which may otherwise change axial location of the spinner head 640 relative to the outlet 625).

The cross-sectional profile of the balloons 678 across the lumen 626 may be minimized such that the balloons 678 do not substantially obstruct the lumen 626 to facilitate material aspirated into the lumen 626. In addition or alternatively, the balloon 678 may engage the inner surface of the catheter 620 when fully inflated to prevent axial movement of the spinner head 640 relative to the catheter 620. For example, the tips of the balloons 678 may include frictional surfaces or materials that prevent the balloons 678 from sliding within the catheter lumen 626 when they are fully inflated. Thus, the balloons 678 may prevent axial migration of the spinner head 640 away from the outlet 625, e.g., due to aspiration forces within the lumen 626.

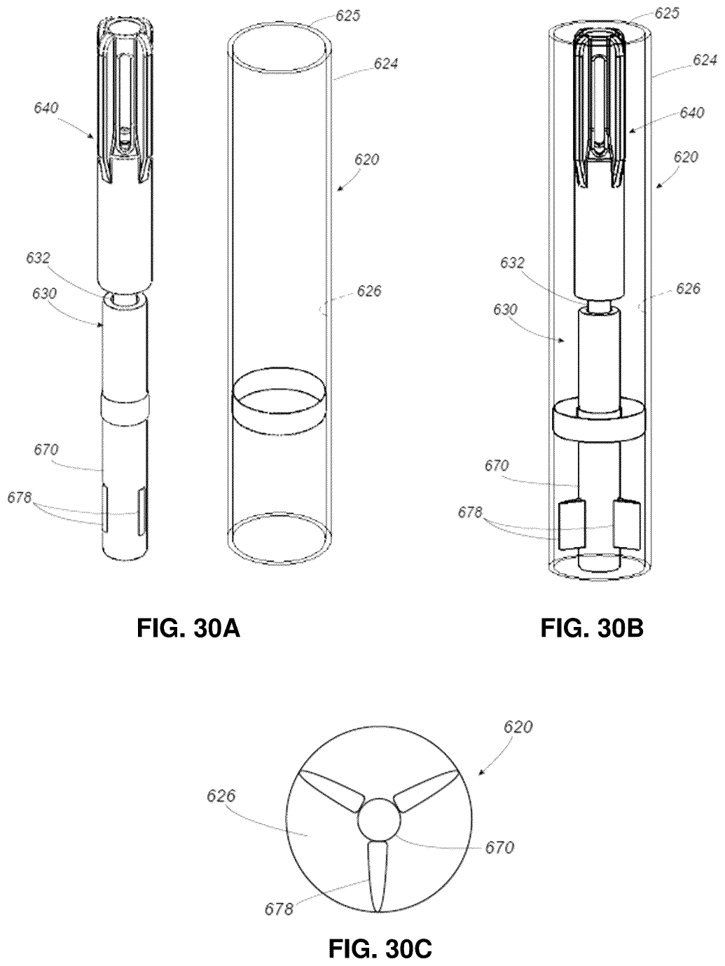

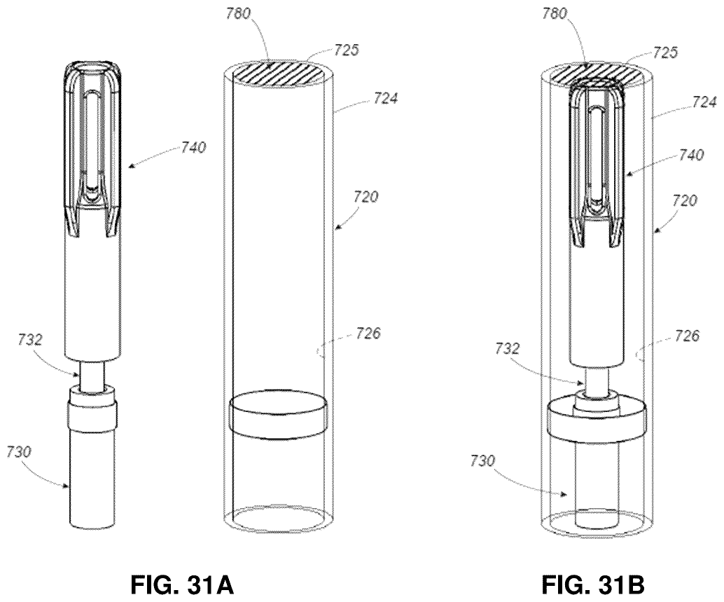

Turning to FIGS. 31A and 31B, in another example, a catheter 720 may be provided that includes a mesh, screen or other obstruction 780 across the outlet 725. The mesh 780 may be sufficiently rigid to prevent the spinner head 740 from being exposed through the outlet 725, e.g., such that the distal end of the spinner head 740 may contact the mesh 780 to prevent further distal movement of the spinner head 740. The open area of the mesh 780 may minimize obstruction of the outlet 725, e.g., to allow material to be aspirated into the lumen 726 through the mesh 780 and/or minimize impact on the localized suction generated when the spinner head 740 rotates adjacent the outlet 725.

Alternatively, the devices and systems herein may include one or more features that axially fix the spinner head at a desired axial position, e.g., immediately adjacent an outlet of a catheter. For example, the spinner device and/or catheter may include a cooperating two-way locking mechanism that axially fixes the spinner head once advanced to a desired position, e.g., to prevent the spinner head from being advanced from the outlet of the catheter and/or inadvertently retracted if suction is applied within the catheter lumen.

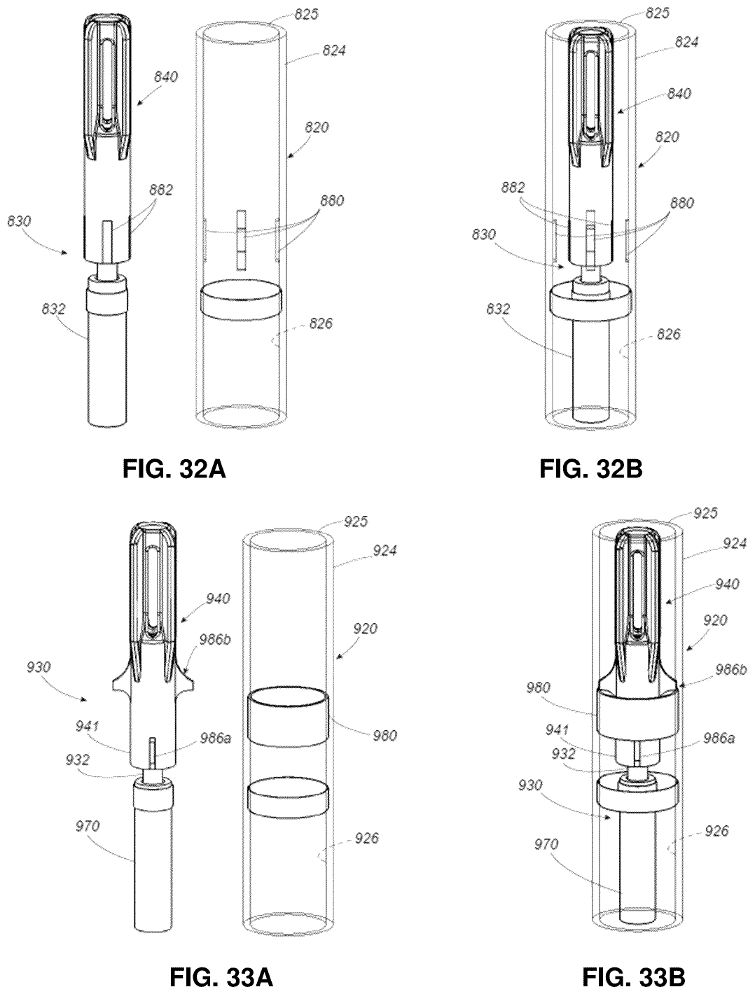

For example, turning to FIGS. 32A and 32B, another exemplary spinner device 830 and aspiration catheter 820 are shown that may be included in the system of FIG. 17. The catheter 820 and spinner device 830 include one or more sets of magnets 880, 882 configured to attract one another to hold the spinner head 840 stationary adjacent the outlet 825 of the catheter 820, e.g., to limit axial movement of the spinner head 840. For example, similar to other examples herein, the spinner device 830 may be slidably received within the lumen 826 of the catheter, e.g., to advance the spinner head 840 towards the outlet.

As best seen in FIG. 32B, the magnets 880, 882 may be attached on the catheter 820 and the spinner device 830 (e.g., on the spinner head 840, shaft 832, or outlet sleeve, not shown) such that the magnets 880, 882 are axially aligned when the spinner head 840 is positioned immediately adjacent the outlet 825. Given the attraction, the magnets 880, 882 may limit further axial movement, e.g., to constrain the spinner head 840 from being exposed out the outlet 825. If it is desired to withdraw the spinner head 840, the spinner device 830 may be pulled with sufficient force to overcome the attraction force between the magnets 880, 882.

Turning to FIGS. 33A and 33B, another two-way locking mechanism is shown that may be provided on a thrombectomy device including an aspiration catheter 920 and a spinner device 930. In this example, the distal end 924 of the catheter 920 includes an internal ring 980 attached within the lumen 926 of the catheter 920 spaced proximally from the outlet 925, similar to other devices herein. However, the spinner device 930 includes a spinner head 940 coupled to a rotation shaft 932 that includes two sets of wings, i.c., a proximal set of wings 986a and a distal set of wings 986b spaced apart axially from one another on a proximal hub 941 of the spinner head 940. The axial spacing of the wings 986a, 986b may correspond to the length of the ring 980, e.g., such that the distal set of wings 986b are positioned just distal to the ring 980 when the proximal set of wings 986a are positioned immediately proximal to the ring 980. Alternatively, the proximal set of wings may be attached or otherwise provided on the shaft 932 or an outer sleeve of the spinner device 930 (not shown).

As shown, the distal wings 986b include tapered edges and/or are sufficiently flexible such that the distal wings 986b may pass through the internal ring 980, e.g., having an outer cross-section larger than an inner diameter of the ring 980, with the tapers allowing the distal wings 986b to be directed through the ring 980. The proximal wings 986a include blunt edges that define an outer cross-section larger than the inner diameter of the ring 980. Consequently, when the spinner device 930 is advanced distally within the lumen 926, the distal wings 986b may initially contact the ring 980 but, when sufficient distal force is applied, the distal wings 986b may pass through the ring 980 until the proximal wings 986a contact the ring 980 and prevent further movement. The distal wings 986b may be sized to limit proximal movement, e.g., until sufficient proximal force is applied to pull the distal wings 986b back through the ring 980, whereupon the spinner device 930 may be retracted further, as desired.

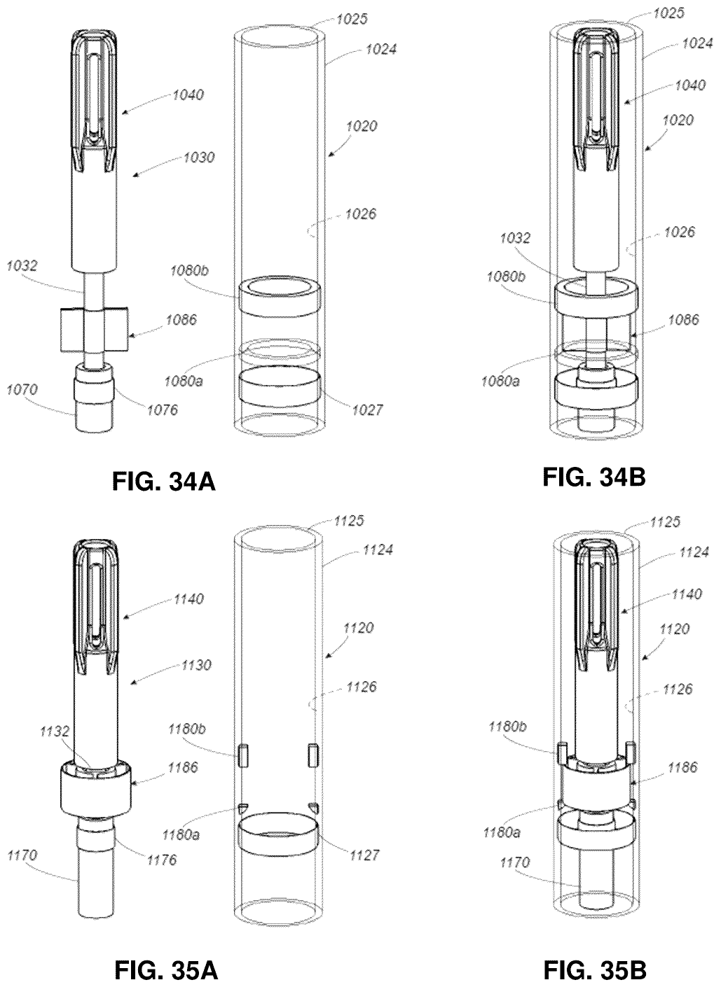

Alternatively, as shown in FIGS. 34A and 34B, two rings or other features 1080a, 1080b may be provided within the catheter 1020 that are spaced axially from one another, and the spinner device 1030 may include a pair of wings or other features 1086 that may be captured between the features 1080a, 1080b to limit axial movement of the spinner device 1030. In the example shown, a proximal ring 1080a may be provided that includes a tapered proximal edge and a blunt or tapered distal edge extending around the inner circumference of the proximal ring 1080a. The proximal ring 1080a is spaced apart proximally from a distal ring 1080b, which may include a blunt proximal edge.

With particular reference to FIG. 34B, the rings 1080a, 1080b are sized such that the spinner head 1040 may pass freely through the rings 1080a, 1080b, and the wings 1086 are sized to interact with the rings 1080a, 1080b to limit distal movement of the spinner head 1040. For example, the tapered proximal edge of the proximal ring 1080a and the wings 1086 may be sized and/or constructed to allow the wings 1086 to directed through the proximal ring 1080a, e.g., when sufficient distal force is applied to the spinner device 1030. Once the wings 1086 pass through the proximal ring 1080a, the wings 1086 may contact the distal ring 1080b, thereby preventing further distal movement of the spinner device 1030. For example, the cross-section of the wings 1086 may be greater than the inner diameter of the distal ring 1080b, e.g., similar to other devices herein. With the wings 1086 positioned between the rings 1080a, 1080b, subsequent axial movement of the spinner device 1030 may be limited, e.g., to prevent the spinner head 1040 from being exposed from the outlet 1025 of the catheter 1020 and being retracted during use, e.g., due to aspiration forces and/or engagement with clot material. Optionally, the wings 1086 may be directed back through the proximal ring 1080a, e.g., if sufficient force is applied, thereby allowing the spinner device 1030 to be directed proximally relative to the catheter 1020.

Turning to FIGS. 35A and 35B, still another example of a thrombectomy device is shown that includes an aspiration catheter 1120 and a spinner device 1130, which may be generally constructed similar to any of the other devices herein. However, in this example, the spinner device 1130 includes a stopper ring 1186 disposed concentrically around to the spinner device 1130, e.g., to the rotation shaft 1132 of the spinner device 1130 proximal to the spinner head 1140. For example, the stopper ring 1186 may be attached to the shaft 1132 by one or more struts or other supports that fix the stopper ring 1186 concentrically around the shaft 1032. Alternatively, the stopper ring may be attached to the spinner head or the outer sleeve, as desired.

The catheter 1120 includes first and second sets of bumps, tabs, or other internal features 1180a, 1180b, e.g., attached within the lumen 1126 of the catheter 1120. For example, as shown, a proximal set of bumps 1180a may be provided, e.g., a plurality of proximal bumps 1180a spaced apart from one another circumferentially at the same axial location, and a plurality of distal bumps 1180b may be spaced apart from one another circumferential distal to the proximal bumps 1180a. The bumps 1180a, 1180b may be sized to allow the spinner head 1140 to through the bumps 1180a, 1180b, but may interact with the stopper ring 1186 to limit axial movement of the spinner head 1140. For example, the proximal bumps 1180a may include tapered edges and/or may be otherwise configured to allow the stopper ring 1186 to pass distally beyond the proximal bumps 1180a, and the distal bumps 1180b am include blunt edges and/or otherwise sized to prevent further distal movement when the stopper ring 1186 contacts the distal bumps 1180b.

Thus, as best seen in FIG. 35B, the bumps 1180a, 1180b may provide a two-way locking mechanism that prevents axial movement of the spinner head 1140 when the stopper ring 1186 is positioned between the sets of bumps 1180a, 1180b. Optionally, the spinner device 1130 may be retracted proximally, e.g., when the shaft 1132 is pulled with sufficient force to direct the stopper ring 1186 proximally past the proximal bumps 1180a.

Any of the devices herein may be used to perform a thrombectomy procedure, e.g., to dissolve and/or reduce clot or other material within a blood vessel or other boy lumen. For example, during use, with reference to FIGS. 18A and 18B, the spinner device 30 (or, similarly, any of the other spinner devices described herein) may be used along with a catheter 20 to perform a thrombectomy procedure. For example, with the spinner head 40 retracted within the catheter 20 proximal to the outlet 25, the distal end 24 of the catheter 20 may be introduced into a patient's body and advanced to a target location, e.g., adjacent a clot within the patient's vasculature (not shown). Once positioned, the shaft 32 may be advanced distally to direct the spinner head 40 towards the distal end 24 of the catheter 20. When the wings 86 of the wing-stopper 82 contact the ring 80 within the catheter 20, further distal movement of the spinner head 40 may be prevented. As explained elsewhere herein, the relative locations of the stops may position the distal end 46 of the spinner head 40 immediately adjacent to or proximal to the outlet 25 of the catheter 20, e.g., to place the distal end 46 immediately adjacent the clot.

The motor 60 may then be activated to rotate the spinner head 40 to dissolve and/or reduce the clot. Optionally, once the clot is treated, the shaft 32 may be actuated to retract the spinner head 40 proximally within the lumen 26 of the catheter 20, and the device 10 may be removed (or directed to one or more additional locations to treat additional clot).

Any of the devices, systems, and methods described herein may optionally include a vacuum source. For example, as shown in FIGS. 17A and 17B, the device 10 may include a source of vacuum 64, e.g., a syringe, suction line, and the like, that may be coupled to the proximal end 22 of the catheter, e.g., to port 52b on the handle 50 for aspirating material into the lumen. In addition or alternatively, a port may be provided that communicates with the interior of the outer sleeve 70, e.g., a port (not shown) on the proximal end 72 of the outer sleeve 70 proximal to the hub 50 of the catheter 20, which may be used to flush and/or aspirate material between the outer sleeve 70 and the rotation shaft 32.

For example, the port 52b may include a Luer fitting or other connectors that allow tubing from the source of vacuum 64 to be removably connected to the port 52b. Before or during advancing and/or activating the spinner head 40 to reduce or dissolve a clot, the vacuum source 64 may be activated (or may be activated immediately upon advancing the spinner head 40, if desired) to aspirate dissolved fibrin or other remaining clot material, e.g., into the lumen 26 of the catheter 20, as described further elsewhere herein. The spinner head 40 may also help reorient and/or reposition the clot relative to the catheter 20, e.g., to enhance contact between the clot and the outlet 25, which may enhance vacuum suction from the lumen 26.

For example, in one sequence of operation, after the catheter 20 is manipulated to position the outlet 25 adjacent a clot, the spinner device 30 may be advanced within the lumen 26 to position the spinner head 40 immediately adjacent the outlet 25 (with the stops or lock mechanisms limiting further movement as described elsewhere herein). The suction may then be activated after the motor is activated to rotate the spinner head 40 or, alternatively, the suction may be activated before rotating the spinner head 40. Once the clot is sufficiently dissolved or reduced, the entire device, i.e., the catheter 20 and spinner device 30 may be retracted proximally to remove the device from the patient's vasculature. The spinner head 40 may continue to be rotated and the suction applied during this retraction to capture and/or withdraw any residual material. Alternatively, the spinner head 40 may be deactivated and the suction may be maintained to prevent residual material from escaping during retraction of the device.

In addition or alternatively, as shown in FIG. 17A, a source of fluid 66, e.g., a syringe of saline, contrast, and the like, may be connected to the port 52b (or to a separate dedicated port, not shown, if desired). Thus, during use of the catheter 20, the lumen 26 may be flushed and/or the fluid may be delivered through the outlet 25, e.g., one or more agents such as tPA, during the procedure, if desired.

Optionally, the thrombectomy device 10 and/or spinner device 30 may be included in a system or kit including one or more additional devices for use during a thrombectomy procedure. For example, the system may include an occlusion device and/or a capture member (not shown) to prevent fragments of a clot being treated from migrating elsewhere within the patient's vasculature. For example, such devices may be introduced and deployed from the catheter 20, e.g., through the lumen 26 or a secondary lumen. Alternatively, such devices may be introduced and deployed independently of the spinner device, e.g., via a separate catheter, sheath, or other device (not shown) downstream from the target clot.

Suitable additional devices for use within a system or kit as described herein may include, for example, a device carrying a balloon or other expandable member may be provided that may be introduced into a body lumen spaced apart from and/or adjacent the spinner head 40 and/or catheter outlet 25, and the expandable member may be expanded to at least partially occlude the body lumen to prevent material from the clot migrating from the body lumen. Alternatively, a capture member may be provided for introduction into a body lumen adjacent the spinner tip, e.g., to capture residual fibrin material from the clot being treated by the spinner tip. Such a capture member may include a filter, a snare, a cage, and the like.

The devices and systems herein may be used during a thrombectomy procedure. For example, the spinner device 30 may be introduced into a body lumen, e.g., a vein, artery, and the like, adjacent a target blood clot, and the spinner head 40 may be rotated to generate localized suction, e.g., to generate localized hydrodynamic forces combining compression and shear forces, to dissolve the clot, reduce clot size, and/or prevent fragmentation of a clot within the body lumen. For example, the spinner device may be deployed to reduce or otherwise dissolve a clot, e.g., by separating the red blood cells from the fibrin and/or other fibrous material. The red blood cells may simply be released within the vessel, e.g., such that the cells are metabolized by the body, and the fibrin may be captured, e.g., directly by the cavity of the spinner device, or using aspiration, a capture device, and the like, and/or treated with a thrombolytic drug or other agent to breakdown, dissolve, and/or otherwise neutralize the residual material before the spinner device is removed from the patient's vasculature.

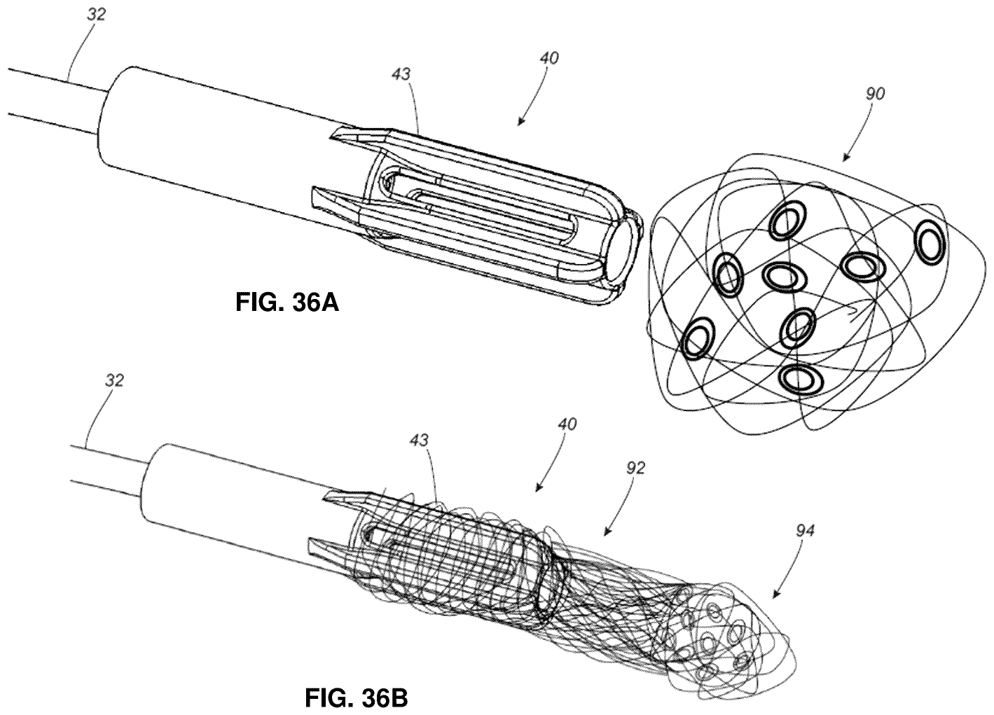

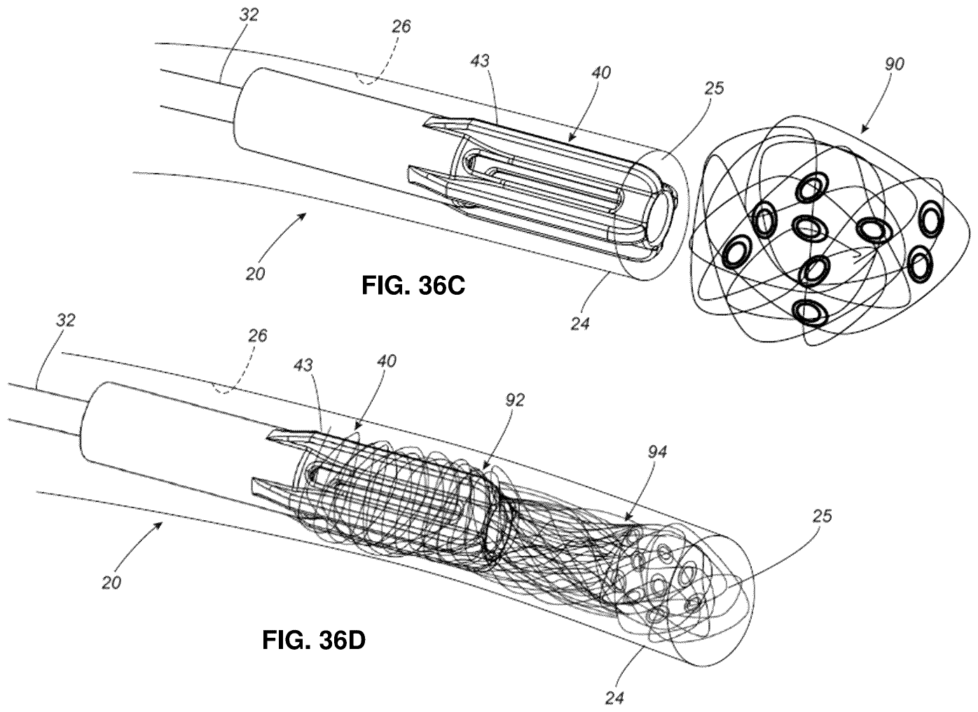

Alternatively, as shown in FIGS. 36A-36D, the spinner head 40 may be used to dissolve or reduce a clot 90 and residual fibrin may be braided or otherwise entangled on the spinner head 40 to mechanically engage the clot 90. For example, the spinner head 40 may be positioned immediately adjacent the clot 90, as shown in FIG. 36A, and then rotated to generate localized suction and the resulting shear forces may separate red blood cells from the clot 90, and then residual fibrin 92 may become braided or entangled around the spinner head 40, as shown in FIG. 36B. Optionally, as shown in FIGS. 36C and 36D, the spinner head 40 may be provided immediately adjacent an outlet 25 of an aspiration catheter 20 and, once the fibrin 92 is braided or entangled around the spinner head 40, the spinner device 30 may be retracted to pull the fibrin into the lumen 26 of the catheter 20. Thus, after at least partially dissolving the clot 90 and the residual fibrin 92 is braided around the spinner head 40, the spinner device 30 may be retracted relative to the catheter 20 to drag the entire remaining clot 94 into the lumen 26 with a firm grasp due to fibrin entanglement and fibrin densification. Alternatively, with sufficient fibrin braided or entangled around the spinner head 40, the remaining clot 94 may remain outside the catheter 20 and pulled proximally along with the spinner head 40 when the catheter 20 and spinner device 30 are subsequently withdrawn from the patient's vasculature together.

Optionally, manipulation of the catheter 20 and/or spinner device 30 may be monitored using external imaging, e.g., fluoroscopy, Xray, ultrasound, and the like. For example, as described elsewhere herein, the spinner device 30 may include one or more radiopaque markers, e.g., markers 76, 27 on the outer sleeve 70 and catheter 20 that may be monitored to facilitate positioning the spinner head 40 adjacent to a clot. Optionally, contrast may be introduced into the blood vessel, e.g., via the port 52b, a separate port on the proximal end 22 of the catheter 20, or a separate device (not shown), to facilitate locating the clot 92 and positioning the spinner head 40, which may be introduced through the catheter 20 (e.g., through the main lumen 26 or a separate lumen) or through a separate device. In addition or alternatively, the catheter 20 and/or spinner device 30 may be monitored using intravascular imaging systems and methods, such as intravascular ultrasound imaging, optical coherence tomography, and the like.

Unlike other thrombectomy devices that attempt to catch or macerate the entire clot (leading to fragmentation of the clot into pieces), the devices and systems of the present disclosure help separate the red blood cells of the clot from the complex fibrin network, thus leading to greater efficiency and efficacy. That is, the spinning motion of the spinner head 40 may generate a shear force which, in combination with the compression from the suction to squeeze out red blood cells trapped in the clot, which may escape through the slits 45 and/or otherwise from the cavity 48 of the spinner head 40 into the vessel. Thus, unlike macerator devices, which mechanical break the clot into pieces and risk fragments traveling to other locations in the patient's body, the devices herein allow the red blood cells to be removed to reduce the volume of the clot without breaking up the residual fibrin material. The residual material may remain substantially intact and then removed, as described elsewhere herein. Further, macerator devices may be incapable of breaking up rich and/stiff fibrin networks of some clots and so may be incapable of removing such clots, while the devices herein allow such residual fibrin network to be captured and/or otherwise removed regardless of the stiffness of the residual material.

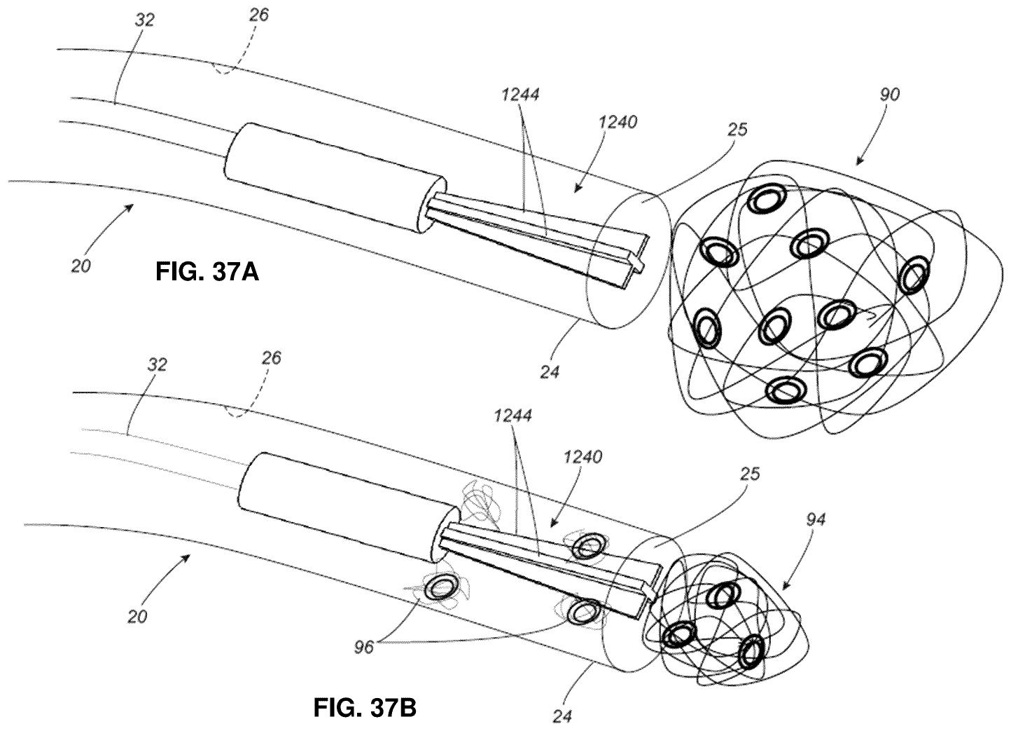

Alternatively, as shown in FIGS. 37 and 37B, a spinner device 1230 may be provided that includes a spinner head 1240 coupled to a rotation shaft 1232, which may be positioned within a lumen 26 of an aspiration catheter 20 immediately adjacent an outlet 25 thereof. In this alternative, the spinner head 1240 includes shredding features, e.g., a plurality of axial struts 1244 arranged around a distal tip of the spinner head 1240. In the example shown, the spinner head 1240 includes four axial struts 1244 extending radially outwardly from a center of the spinner head 1240, thereby defining a cross-shaped cross-section. Alternatively, the spinner head may include fewer struts, e.g., two or three struts, or more struts, e.g., five, six, or more struts, as desired. The struts 1244 may generate localized suction to dissolve or reduce a clot and/or may shred or otherwise break residual material, e.g., the residual fibrin network of the clot, into pieces, which may be aspirated into the lumen 26 of the catheter 20.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

What is claimed is:

1. A device for performing thrombectomy procedures, the device comprising:
a tubular member including a proximal end, a distal end sized for introduction into a body lumen and then advanced to a point that is adjacent clot, and a lumen extending from the proximal end to an outlet in the distal end;
a spinner device comprising a rotation shaft comprising a shaft proximal end configured to be coupled to a motor to spin the shaft, a shaft distal end carrying a spinner head movable axially within the lumen, the shaft configured to rotate the spinner head and the spinner head configured to generate localized suction adjacent the outlet when the shaft rotates to reduce clot size of clot within a body lumen adjacent the outlet; and
a one or more stops configured to prevent the spinner tip from extending beyond the distal end opening of the tubular member.

2. The device of claim 1, wherein the one or more stops comprises cooperating stops on the spinner device and tubular member configured to limit advancement of the spinner device to prevent the spinner head from being exposed beyond the distal end of the tubular member.

3. The device of claim 1, wherein the spinner head comprises a hollow body having a distal opening, one or more external projections extending from a lateral side of the hollow body, and one or more openings into the hollow body along the lateral side of the hollow body.

4. The device of claim 1, further comprising an actuator for directing the shaft axially within the lumen to position the spinner tip adjacent the outlet.

5. The device of claim 1, further comprising an outer sleeve surrounding the rotation shaft at least partially between the shaft proximal end and the shaft distal end.

6. The device of claim 5, further comprising a port on the proximal end of the outer sleeve configured to be connected to a source of vacuum or fluid, the port communicating with a lumen within the outer sleeve extending to a distal end of the outer sleeve.

7. The device of claim 1, further comprising a port on the proximal end of the tubular member configured to be connected to a source of vacuum, the port communicating with the lumen to generate suction into the outlet.

8. The device of claim 1, further comprising one or more radiopaque markers on the spinner device and the tubular member that are axially aligned when the spinner head is positioned adjacent the outlet.

9. The device of claim 8, wherein the one or more radiopaque markers comprise a marker band on the distal end of the tubular member offset proximally from the outlet.

10. The device of claim 9, further comprising an outer sleeve surrounding the rotation shaft at least partially between the shaft proximal end and the shaft distal end, and wherein the one or more radiopaque markers comprise a marker band on a distal end of the outer sleeve that is axially aligned with the marker band on the tubular member when the spinner head is positioned immediately adjacent the outlet.

11. The device of claim 2, wherein the one or more cooperating stops comprise:
an internal ring on the tubular member within the lumen; and
a plurality of wings extending radially outwardly from the outer sleeve and sized to contact the internal ring when the spinner head is positioned immediately adjacent the outlet to prevent further distal movement of the spinner head.

12. The device of claim 11, wherein the plurality of wings are fixed on the distal end of the outer sleeve.

13. The device of claim 11, wherein the plurality of wings extend radially outwardly from a circular base attached to the distal end of the outer sleeve.

14. The device of claim 2, wherein the one or more cooperating stops comprise: an internal ring on the tubular member within the lumen; and a plurality of wings extending radially outwardly from the rotation shaft.

15. The device of claim 14, wherein the plurality of wings extend radially outwardly from a circular base attached to the rotation shaft proximal to the spinner head.

16. The device of claim 1, wherein the one or more cooperating stops comprise: an internal ring on the tubular member within the lumen; and a plurality of wings extending radially outwardly from the spinner head.

17. The device of claim 16, wherein the plurality of wings extend radially outwardly from a proximal end of the spinner head, the spinner head comprising one or more features distal to the plurality of wings configured to generate the localized suction when the spinner head is rotated by the rotation shaft.

18. The device of claim 2, wherein the one or more cooperating stops comprise:
an internal ring on the tubular member within the lumen; and
a plurality of wings extending radially outwardly from a ring free-floating on the rotation shaft adjacent the spinner head.

19. The device of claim 18, further comprising an outer sleeve surrounding the rotation shaft at least partially between the shaft proximal end and the shaft distal end, the outer sleeve comprising an outer sleeve distal end axially fixed adjacent the spinner head, and wherein the ring is positioned between the spinner head and the outer sleeve distal end.

20. The device of claim 19, further comprising a blocker on the rotation shaft between the ring and the outer sleeve distal end.

21. The device of claim 20, wherein the blocker comprises an annular body fixed on the rotation shaft.

22. The device of claim 20, wherein the plurality of wings comprise tapered proximal edges configured to contact an outer distal edge of the blocker to reduce friction between the plurality of wings and the blocker.

23. A device for performing thrombectomy procedures, comprising:
a tubular member including a proximal end, a distal end sized for introduction into a body lumen adjacent clot, a lumen extending from the proximal end to an outlet in the distal end, and an annular stop within the lumen and fixed at a location proximal to the outlet;
a spinner device comprising:
a rotation shaft comprising a shaft proximal end configured to be coupled to a motor to spin the shaft and a shaft distal end sized for introduction into the lumen;
an outer sleeve surrounding at least a portion of the rotation shaft, the outer sleeve comprising an outer sleeve distal end adjacent the shaft distal end;
a spinner head carried on the shaft distal end, the shaft movable axially within the lumen to position the spinner head adjacent the outlet, the shaft configured to rotate the spinner head to generate localized suction adjacent the outlet when the shaft rotates to reduce clot size of clot within a body lumen adjacent the outlet; and
a plurality of wings fixed to and extending radially outwardly from the outer sleeve distal end and sized to contact the annular stop when the spinner head is positioned immediately adjacent the outlet to prevent further distal movement of the spinner head.

24. A method for performing thrombectomy, comprising:
introducing a distal end of a tubular member into a body lumen to position an outlet of the tubular body communicating with a lumen of the tubular member adjacent a target blood clot;
advancing a spinner head of a spinner device within the lumen until the spinner head is positioned adjacent the outlet, the spinner device and tubular member distal end comprising cooperating stops that prevent the spinner head from being exposed beyond the outlet, and
rotating the spinner device to generate localized suction, e.g., to generate compression and/or shear forces, to dissolve the clot, to separate red blood cells from fibrin, to reduce clot size, and/or to prevent fragmentation of the clot.
